SEQUENCE LISTING (20 sequences)

SEQ ID NO: 1 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 2 - protein, 585 aa, Pan troglodytes
MTPPRLFWVWLLVAGTQGVNDGDMRLADGGATNQGRVEIFYRGQWGTVCDNLWDLTDASVVCRALGFENATQALGRAAFGQGSGPIMLDEVQCMGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPRDPSFQTPLDLYAYAVATGDALLEKLCLQFLAWNFEALTQAEAWPSVPTDLLQLLLPRSDLAVPSELALLKAVDTWSWGERASHEEVEDLVEKIRFPMMLPEELFELQFNLSLYWSHEALFQKKTLQALEFHTVPFQLLARYKGLNLTEDTYKPRIYTSPTWSASVTDSSWSARKSQLVYQSRRGPLVKYSSNYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDINSSKSTSSFPCPAGHFNGFRTVIRPFYLTNSSGVD

SEQ ID NO: 3 - protein, 559 aa, Canis lupus famiilaris
MALPLVLWMCLLVAGTQGVKDGDMRLANGDTANEGRVEIFYSGRWGTVCDNLWDLMDASVVCRALGFENATEALGGAAFGPGKGPIMLDEVECTGTEPSLANCTSLGWMKSNCRHNQDAGVVCSNETRGAHTLDLSGELPAALEQIFDSQRGCDLSIRVKVKDQEEEGPHFCAHRLILAANPEAQALCKAPGSTVTMEVDAECLPVVRDFIRYLYSRRLDISLTSVKCFHKLASAYEAQQLQSFCASLFAILLPEDPSFQAPLDLYAYALATQDPVLEELCVQFLAWNFEGLTQATAWPRVPTALLQLLLSRSELAVPSELALLTALDVWSQERRPSHGEVARLVDKVRFPMMLPEHLFELQFNLSLYWSHEALFQKKILQALEFHTVPFRLLAQHRGLNLTEDAYQPRLYTSPTWSASVSRSSSRYWNYPYQSFQTPQHPSFLFQNKYISWSLVYLPTVQSCWNYGFSCSSDEVPLLGLSKSDYSDPTIGYENKALMRCGGRFVADVTDFEGQKALIPSALGTNSSRRPSLFPCLGGSFSSFQVVIRPFYLTNSSDVD

SEQ ID NO: 4 - protein, 555 aa, Bos taurus
MAPLRLFWIWLLVVGTRGVKDGDMRLADGGSANQGRVEIYYNGQWGTVCENMWDLTDASVVCRALGFQNATEALGGAAFGPGYGPIMLDEVRCTGTEPSLANCSSLGWMRSNCRHDKDASVICTNETRGVYTLDLSGELPAALEQIFESQKGCDLFITVKVREEDEIAMCAHKLILSTNPEAHGLWKEPGSRVTMEVDAECVPVVKDFIRYLYSRRIDVSLSSVKCLHKFASAYQAKQLQSYCGHLFAILIPQDPSFWTPLELYAYALATRDTVLEEICVQFLAWNFGALTQAEAWPSVPPALLQGLLSRTELVVPSELVLLLAVDKWSQERHTSHKEVEALVGQVRFPMMPPQDLFSLQFNLSLYWSHEALFQKKILQALEFHTVPFELLAQYWGLNLTEGTYQPRLYTSPTWSQSVMSSSYNPSRSFQTPQHPSFLFHDSSVSWSFVYLPTLQSCWNYGFSCSSDDPPLLALSKSSYSKSNPTIGYENRALLHCEGSFVVDVIDFKGWKALVPSALATNSSRSTSLFPCPSGVFSRFQVVIRPFYLTNSTDMD

SEQ ID NO: 5 - protein, 577 aa, Mus musculus
MALLWLLSVFLLVPGTQGTEDGDMRLVNGASANEGRVEIFYRGRWGTVCDNLWNLLDAHVVCRALGYENATQALGRAAFGPGKGPIMLDEVECTGTESSLASCRSLGWMVSRCGHEKDAGVVCSNDTTGLHILDLSGELSDALGQIFDSQQGCDLFIQVTGQGYEDLSLCAHTLILRTNPEAQALWQVVGSSVIMRVDAECMPVVRDFLRYFYSRRIEVSMSSVKCLHKLASAYGATELQDYCGRLFATLLPQDPTFHTPLDLYAYARATGDSMLEDLCVQFLAWNFEPLTQSESWSAVPTTLIQALLPKSELAVSSELDLLKAVDQWSTETIASHEDIERLVEQVRFPMMLPQELFELQFNLSLYQDHQALFQRKTMQALEFHTVPVEVLAKYKGLNLTEDTYKPRLYTSSTWSSLVMASTWRAQRYEYNRYNQLYTYGYGSVARYNSYQSFQTPQHPSFLFKDKQISWSATYLPTMQSCWNYGFSCTSNELPVLGLTTSSYSNPTIGYENRVLILCGGYSVVDVTSFEGSKAPIPTALDTNSSKTPSLFPCASGAFSSFRVVIRPFYLTNSTDMV

SEQ ID NO: 6 - protein, 574 aa, Ratus norvegicus
MALLWLLSVFLLVPGTQGAKDGDMRLVNGASASEGRVEIFYRGRWGTVCDNLWNLLDAHVVCRALGYENATQALSRAAFGPGKGPIMLDEVECTGNESSLANCSSLGWMVSHCGHEKDAGVVCSNDSRGIHILDLSGELPDALGQIFDSQQDCDLFIQVTGQGHGDLSLCAHTLILRTNPEAQALWQVVGSSVIMRVDAECMPVVRDFLRYFYSRRIEVSMSSVKCLHKLASAYGATELQGYCGRLFVTLLPQDPTFHTPLELYEYAQATGDSVLEDLCVQFLAWNFEPLTQAEAWLSVPNALIQALLPKSELAVSSELDLLKAVDQWSTATGASHGDVERLVEQIRFPMMLPQELFELQFNLSLYQGHQALFQRKTMEALEFHTVPLKVLAKYRSLNLTEDVYKPRLYTSSTWSSLLMAGAWSTQSYKYRQFYTYNYGSQSRYSSYQNFQTPQHPSFLFKDKLISWSATYLPTIQSCWNYGFSCTSDELPVLGLTTSSYSDPTIGYENKALILCGGYSVVDVTTFIGSKAPIPGTQETNSSKTPSLFPCASGAFSSFREVIRPFYLTNSTDTE

SEQ ID NO: 7 - DNA, 20 bp, Artificial Sequence
  Description of Artificial Sequence Forward Primer
ggctcatgaccacagtccat

SEQ ID NO: 8 - DNA, 19 bp, Artificial Sequence
  Description of Artificial Sequence Reverse Primer
gcctgcttcaccaccttct

SEQ ID NO: 9 - DNA, 22 bp, Artificial Sequence
  Description of Artificial Sequence Forward Primer
gccaaggaaaatgtaacccagg

SEQ ID NO: 10 - DNA, 23 bp, Artificial Sequence
  Description of Artificial Sequence Reverse Primer
gcctctgttccaactgatagtga

SEQ ID NO: 11 - DNA, 21 bp, Artificial Sequence
  Description of Artificial Sequence Forward Primer
gcagtgggatcactttcacaa

SEQ ID NO: 12 - DNA, 21 bp, Artificial Sequence
  Description of Artificial Sequence Reverse Primer
cttgaagggaagggctgtttt

SEQ ID NO: 13 - DNA, 21 bp, Artificial Sequence
  Description of Artificial Sequence Forward Primer
aactctacatctcccttcccg

SEQ ID NO: 14 - DNA, 20 bp, Artificial Sequence
  Description of Artificial Sequence Reverse Primer
ctcctgtcgcatgtcactcc

SEQ ID NO: 15 - DNA, 19 bp, Artificial Sequence
  Description of Artificial Sequence Forward Primer
acgcaggggtggacaaaac

SEQ ID NO: 16 - DNA, 20 bp, Artificial Sequence
  Description of Artificial Sequence Reverse Primer
gagacacacaccgacttggg

SEQ ID NO: 17 - DNA, 27 bp, Artificial Sequence
  Description of Artificial Sequence Sense Primer
tagacatatgacccctccgaggctctt

SEQ ID NO: 18 - DNA, 55 bp, Artificial Sequence
  Description of Artificial Sequence Antisense Primer
atcaggatccttacttgtcatcgtcgtccttgtagtcgtccacacctgaggagtt

SEQ ID NO: 19 - DNA, 29 bp, Artificial Sequence
  Description of Artificial Sequence Sense Primer
gacacatatggctcttctgtggctcctct

SEQ ID NO: 20 - DNA, 31 bp, Artificial Sequence
  Description of Artificial Sequence Antisense Primer
tcggatccttagttggtgaggtagaaggggc

(19) United States
(12) Patent Application Publication
(10) Pub. No.: US 2012/0121590 A1
(43) Pub. Date: May 17, 2012

(54) METHODS OF INHIBITING ADVERSE CARDIAC EVENTS AND TREATING ATHEROSCLEROSIS AND CORONARY ARTERY DISEASE USING GALECTIN-3 BINDING PROTEIN (GAL-3BP, BTBD17B, MAC-2 BINDING PROTEIN)

(76) Inventors: Klaus Ley, La Jolla, CA (US); Christian Gleissner, Heidelberg (DE)

(21) Appl. No.: 13/256,899

(22) PCT Filed: Apr. 16, 2010
(86) PCT No.: PCT/US10/31479

§ 371 Date: Jan. 31, 2012

Related U.S. Application Data
(60) Provisional application No. 61/170,277, filed on Apr. 17, 2009.

(51) Int. Cl. A61K 39/395; G01N 33/566; A61K 38/00; A61P 9/10; C07K 14/435; C07K 7/06; C07K 7/08; A61P 9/00; C12Q 1/68; A61K 38/17

(52) U.S. Cl. 424/134.1; 435/6.12; 435/7.92; 436/501; 514/1.1; 514/16.4; 514/21.3; 530/324; 530/325; 530/326; 530/327; 530/328; 530/329; 530/330; 530/350

(57) ABSTRACT

The invention provides Galectin-3 binding protein (Gal-3BP, BTBD17B) polypeptide sequences and compositions that include Gal-3BP polypeptide sequences, and methods of using Gal-3BP polypeptide sequences, including modified forms and wild type (native) forms of Gal-3BP polypeptide, such as in treatment, diagnostic, detection and prognostic methods.

### RELATED APPLICATIONS

This application claims the benefit of priority of application Ser. No. 61/170,277, filed Apr. 17, 2009, which is expressly incorporated herein by reference in its entirety.

### STATEMENT AS TO FEDERALLY SPONSORED RESEARCH

This invention was made with Government support under Grant No. HL058108 awarded by the National Institutes of Health. The Government has certain rights in this invention.

### TECHNICAL FIELD

The invention relates to Galectin-3 binding protein (Gal-3BP, BTBD17B) polypeptides, and methods of use, for example, in treatment of adverse cardiovascular events and artherosclerotic plaque formation.

### Introduction

Atherosclerosis is an inflammatory disease of the arterial wall characterized by monocytes entering the subendothelial space where they differentiate into macrophages and foam cells (Lusis, Nature 407: 233 (2000); Glass & Witztum, Cell 104: 503 (2001); and Galkina & Ley, Annu. Rev. Immunol. 27: 165 (2009); Li & Glass, Nat. Med. 8: 1235 (2002)). Foam cell formation induced by oxidized low density lipoprotein (oxLDL) leads to induction of pro-inflammatory factors that initiate plaque formation and finally plaque rupture with deleterious clinical consequences like myocardial infarction or stroke. oxLDL-induced foam cell formation is promoted by scavenger receptors like CD36 and SR-A, which allow uncontrolled accumulation of modified LDL cholesterol in foam cells (Libby et al., Am. J. Med. 104: 14S (1998); and Kunjathoor et al., J. Biol. Chem. 277: 49982 (2002)).

### SUMMARY

The invention provides methods of reducing or decreasing risk of adverse cardiovascular events and cardiovascular diseases. In one embodiment, a method includes administering a Galectin-3 binding protein (Gal-3BP, BTBD17B) polypeptide to a subject in an amount that increases Gal-3BP polypeptide in the subject thereby reducing or decreasing risk of the adverse cardiovascular event or cardiovascular disease in the subject. In various non-limiting aspects, a cardiovascular disease is coronary artery disease, peripheral artery disease, cerebrovascular disease, or renal artery disease. In various non-limiting aspects, an adverse cardiovascular event is a stroke, myocardial infarction (heart attack), ischemic heart failure, transient ischemic attack or brain trauma.

The invention also provides methods of reducing or decreasing risk of artherosclerotic plaque formation. In one embodiment, a method includes administering a Galectin-3 binding protein (Gal-3BP) polypeptide to a subject in an amount that increases Gal-3BP polypeptide in the subject thereby reducing or decreasing risk of artherosclerotic plaque formation in the subject.

The invention additionally provides methods of reducing or inhibiting artherosclerotic plaque formation. In one embodiment, a method includes administering a Galectin-3 binding protein (Gal-3BP) polypeptide to a subject in an amount that increases Gal-3BP polypeptide in the subject thereby reducing or inhibiting artherosclerotic plaque formation in the subject.

The invention further provides methods of reducing or inhibiting foam cell formation. In one embodiment, a method includes administering a Galectin-3 binding protein (Gal-3BP) polypeptide to a subject in an amount that increases Gal-3BP polypeptide in the subject thereby inhibiting or reducing foam cell formation in the subject.

The invention moreover provides methods of increasing or stimulating Galectin-3 binding protein (Gal-3BP) polypeptide levels. In one embodiment, a method includes administering to the subject an amount of a compound that increases or stimulates Gal-3BP polypeptide levels in the subject.

Methods of the invention include increasing Gal-3BP polypeptide to an amount greater than prior to administration. In particular embodiments, Gal-3BP polypeptide increases to an amount in the subject of greater than 2 ug/ml in blood plasma, increases to an amount greater than 5 ug/ml in blood plasma, greater than 10 ug/ml in blood plasma, greater than 15 ug/ml in blood plasma, or greater than 20 ug/ml in blood plasma.

Methods of the invention also include increasing Gal-3BP polypeptide to an amount greater than prior to administration for a period of time greater than 12, 24, 36, 48, 72 hours, or 3, 4, 5, 6, 7, 8, 9, 10, 12, 14 days, weeks or months.

The invention still moreover provides methods of diagnosing a subject having or at increased risk of having an adverse cardiovascular event or cardiovascular disease, artherosclerotic plaque formation, foam cells or foam cell formation. In one embodiment, a method includes contacting a biological material or sample from a subject with an agent that binds to Gal-3BP polypeptide sequence and assaying for the amount of Gal-3BP polypeptide, wherein an amount less than about 10 ug/ml diagnoses the subject as having or at increased risk of developing an adverse cardiovascular event, cardiovascular disease, artherosclerotic plaque formation, foam cells or foam cell formation. In particular aspects, the agent is an antibody that binds to Gal-3BP polypeptide or a nucleic acid that hybridizes to a nucleic acid encoding Gal-3BP polypeptide sequence. Exemplary cardiovascular diseases include, without limitation, coronary artery disease, peripheral artery disease, cerebrovascular disease, or renal artery disease. Exemplary adverse cardiovascular events include, without limitation, stroke, myocardial infarction (heart attack), ischemic heart failure, transient ischemic attack or brain trauma.

Gal-3BP polypeptides useful in the methods include mammalian forms, such as human. Additional forms include other primates (e.g., Pan troglodytes), dogs (e.g., Canis lupus familiaris), cattle (Bos Taurus), and rodents (e.g., Mus musculus and Rattus norvegicus).

Gal-3BP polypeptide useful in the methods include full length Gal-3BP polypeptide, as well as modified forms of Gal-3BP polypeptide, such as fragments and chimeras and fusions. Non-limiting exemplary polypeptide fragments of Gal-3BP polypeptide include all or a portion of residues 24-124 (SRCR domain); residues 153-221 (BTB domain); or residues 260-360 (BACK domain).

Candidate subjects for methods of the invention, including treatment, diagnostic and prognostic methods include subjects that have or are at increased risk of an adverse cardiovascular event or cardiovascular disease, such as an acute or chronic adverse cardiovascular event or cardiovascular disease. Candidate subjects for methods of the invention, including treatment, diagnostic and prognostic methods also include subjects that have a blood plasma level of Gal-3BP polypeptide less than 10 ug/ml prior to administration, or less than 5 ug/ml prior to administration, or less than 3 ug/ml prior to administration. Candidate subjects for methods of the invention, including treatment, diagnostic and prognostic methods additionally include subjects that have a blood plasma levels of greater than 100 mg/dL total cholesterol prior to administration (e.g., greater than 150 mg/dL total cholesterol, or greater than 200 mg/dL total cholesterol, or greater than 250 mg/dL total cholesterol prior to administration). Candidate subjects for methods of the invention, including treatment, diagnostic and prognostic methods further include subjects with a plasma total cholesterol level of less than 200 mg/dL prior to administration, or less than 100 mg/dL prior to administration, or a plasma triglyceride level of greater than 150 mg/dL prior to administration. Candidate subjects for methods of the invention, including treatment, diagnostic and prognostic methods moreover include subjects with a blood plasma level of C reactive protein (CRP) greater than 1 ug/ml prior to administration. Further subjects include, without limitation, a subject that is undergoing or is a candidate for a blood cholesterol lowering therapy, or for treatment with a statin, ACE inhibitor, calcium antagonist, anti-diabetic, or beta-blocker.

The invention still further provides modified forms, such as subsequences of full length Galectin-3 binding protein (Gal-3BP) polypeptide, that have a function or activity of unmodified (e.g., full length) Galectin-3 binding protein. In various embodiments, a modified form (e.g., a subsequence) inhibits, reduces, decreases or suppresses foam cell formation, macrophage cell expression of scavenger receptor A and/or CD36, macrophage accumulation or uptake of modified LDL (e.g., ox LDL or otherwise modified LDL), plaque formation, formation of atherosclerotic lesions or development of atherosclerosis, or increases, promotes or induces secretion of IL-2. In particular aspects, a Galectin-3 binding protein (Gal-3BP) subsequence consists of Gal-313P residues 24-124 (SRCR domain); residues 153-221 (BTB domain); residues 260-360 (BACK domain), or a subsequence of Gal-3BP residues 24-124 (SRCR domain); residues 153-221 (BTB domain); residues 260-360 (BACK domain), or is about 5-10, 10-20, 20-50, 50-75 or 50-100 amino acids in length and includes all or a portion of Gal-3BP residues 24-124 (SRCR domain); residues 153-221 (BTB domain); or residues 260-360 (BACK domain). Non-limiting additional subsequences of Galectin-3 binding protein (Gal-3BP) polypeptide are about 5-10, 10-20, 20-50, 50-100, 100-150, 150-200, 200-250, 250-300, 300-350, 350-400, 400-500, 500-600 or more amino acids in length, and less than full length BTBD17B polypeptide sequence.

Such modified forms of Galectin-3 binding protein (Gal-3BP) (e.g., a subsequences) include isolated and purified forms.

The invention still additionally provides pharmaceutical compositions. In one embodiment, a pharmaceutical composition includes Gal-3BP polypeptide (e.g., full length) and a pharmaceutically acceptable carrier (e.g., such as saline). In another embodiment, a pharmaceutical composition includes a modified Galectin-3 binding protein (e.g., a subsequence of full length Galectin-3 binding protein) that inhibits, reduces, decreases or suppresses foam cell formation, macrophage cell expression of scavenger receptor A and/or CD36, macrophage accumulation or uptake of modified LDL (e.g., ox LDL or otherwise modified LDL), plaque formation, formation of atherosclerotic lesions or development of atherosclerosis, or increases, promotes or induces secretion of IL-2.

Such compositions can include Gal-3BP polypeptide (or a modified from) in any amount. Non-limiting amounts include Gal-3BP polypeptide at a concentration of about 1 mg/ml, or in a range of about 100 μg/ml to 10 mg/ml, in an amount of 10-100 milligrams, or in an amount of between about 1-50 milligrams.

### DESCRIPTION OF THE DRAWINGS

FIGS. 1A-1C show monocyte, macrophage, and foam cell expression data and peptide coverage of Gal-3BP. (A) Standardized heatmap of monocyte macrophage surface receptor gene expression during monocyte-macrophage-foam cell differentiation. Peripheral blood monocytes were differentiated with M-CSF for six days and then exposed to 100 μg/ml oxLDL for two additional days to investigate gene expression in monocytes, macrophages and foam cells. LGALS3BP gene expression is highlighted by a yellow box. Gene expression was determined by Affymetrix gene chip analysis. Expression data are presented in Table 2. (B) Bar graph of LGALS3BP gene expression in monocytes, macrophages and foam cells as determined by Affymetrix gene chip analysis depicted in (A). P<0.0001 for all conditions as determined by heterogeneous error map (HEM) analysis, * P<0.05 between monocytes and macrophages, *** P<0.001 between macrophages and foam cells as determined by local pooled error (LPE) test, n=2. (C) Peptide coverage of Gal-3BP identified in microparticles by tandem mass spectrometry (MS²). Color intensity corresponds to the frequency of detection across all samples (n=13).

FIGS. 2A-2P show that Gal-3BP downregulates scavenger receptor expression in human primary monocyte-derived macrophages. Primary human monocyte-derived macrophages were exposed to human recombinant Gal-3BP (10 μg/ml) and mRNA expression of the scavenger receptors CD36 and scavenger receptor-A (SR-A) was measured real-time PCR (A,B). CD36 (C) and SR-A (D) surface expression was measured by flow cytometry and expressed relative to vehicle control (E,F). Gal-3pb (5 μg/ml) significantly downregulated CD36 (G) and SR-A (H) as early as 6 hours after treatment. Dose response studies showed a maximum effect at a concentration of 10 μg/ml (I,J). Immunofluorescence images of primary human monocyte-derived macrophages treated with vehicle (K) or recombinant human Gal-3BP (10 mg/ml, L) for 24 hours, then exposed to DiI-labeled (red) acetylated LDL (acLDL; 10 mg/ml) for 4 hours. Representative histograms of acLDL (M) or oxLDL (N, 4 hours, Gal-3BP concentration indicated in μg/ml, pre-treatment 24 hrs). Results of 3 to 5 independent experiments are summarized as bar graphs (O,P; MFI mean fluorescence intensity). *P<0.05, **P<0.01, ***P<0.001

FIGS. 3A-3J show the effect of Gal-3BP on CD36 expression and LDL uptake in peritoneal and aortic macrophages. Bone marrow cells were harvested from wild-type and Lgals3bp^{-/-} mice, differentiated with GM-CSF (8 days, 30 ng/ml), LPS (100 ng/ml) and IFN-γ (250 units/ml) for 24 hrs to produce M1 macrophages. Surface expression of CD11b and CD36 was measured by flow cytometry (a) and CD36 expressing cells were counted and expressed as percent of all CD11b+ cells (b, n=3 studies, * P<0.05 by paired T test). Effects of Gal-3BP on foam cell formation in vivo were assessed by injecting wild type mice with thioglycollate intraperitoneally followed by DiI-labeled acLDL with or without recombinant murine Gal-3BP (2.3 μg/mouse) at 48 hours. At 72 hours, cells were harvested, counted (c), and analyzed by flow cytometry for expression of CD11b and GR1 (d) as well as DiI-acLDL uptake (e,f). In two separate studies (n=5 mice per group), wild-type and Lgals3bp^{-/-} mice were injected with DiI-oxLDL i.p. (50 μl of 200 μg/ml solution) and peritoneal cells counted after lavage at 24 hrs (g). In separate experiments, wild-type and Lgals3bp^{-/-} mice (n=5 each) were injected with DiI-oxLDL i.v., aortas were harvested at 24 hrs and processed as described (Galkina, E. et al., J. Exp. Med. 203:1273 (2006)). CD11b expression and oxLDL uptake were measured in live CD45⁺ leukocytes by flow cytometry (h,i) and data expressed as percent oxLDL⁺ cells among all CD11b⁺ cells (j). * P<0.05, **P<0.01.

FIGS. 4A-4E show that plasma Gal-3BP increases with age and is associated with reduced adverse events in patients with coronary artery disease. Gal-3BP plasma levels in healthy controls (n=23, age 24 to 32), patients with angiographically determined coronary artery disease (CAD, n=77, age 38 to 71) and age-matched controls (n=17, age 41-83) as determined by ELISA (a). Gal-3BP plasma levels of 77 patients with angiographically confirmed obstructive CAD with or without major adverse cardiac events (MACE) as defined by death, myocardial infarction, cerebrovascular incident, or the need for surgical or percutaneous coronary revascularization within one year were measured by ELISA. P=0.024 as determined by Mann Whitney test (b). Kaplan Meier analysis of event-free survival in patients with Gal-3BP plasma levels <8.8 or ≧8.8 μg/ml, which was the best cut-off as determined by receiver operator characteristics analysis. P=0.029 as determined by log rank test (c). Data were re-analyzed for a more stringent end point (cardiovascular death, myocardial infarction, cerebrovascular incident within one year); P=0.033 as determined by Mann Whitney test (d). For Kaplan Meier analysis (e) of event-free survival, 8.7 μg/ml was the best cut-off as determined by receiver operator characteristics analysis; P=0.007 as determined by log rank test.

FIGS. 5A-5B show Gal-3BP expression within human atherosclerotic lesions. (A) Human coronary arteries from patients with coronary artery disease were co-stained post mortem for smooth muscle α-actin (Texas red) and Gal-3BP (FITC, green). DAPI (blue) as nuclear stain. (B) Human coronary arteries from patients with atherosclerotic disease were co-stained post mortem for CD68 (Texas red) and Gal-3BP (FITC, green). DAPI (blue) as nuclear stain.

FIG. 6 shows that Gal-3BP and high sensitivity C-reactive protein (hs-CRP) plasma levels are not correlated. Dot plot of Gal-3BP versus hs-CRP plasma levels in the 77 patients with obstructive coronary artery disease. r=0.124, P=0.285 as determined by non-parametric correlation analysis (Spearman).

FIGS. 7A-7B show Gal-3BP expression within human atherosclerotic lesions. (A) and (B) Human coronary arteries from patients with coronary artery disease were stained post mortem for CD68 and Gal-3BP as indicated. Staining was visualized with DAB, sections were counterstained with hematoxylin.

FIGS. 8A-8D show the effect of Gal-3BP on foam cell formation in vivo. Effects of Gal-38P on foam cell formation in vivo were assessed by injecting wild type mice with thioglycollate intraperitoneally followed by DiI-labeled acLDL with or without recombinant murine Gal-3BP (2.3 μg/mouse) at 0 hours (when thioglycollate was given) or at 48 hours. At 72 hours, cells were harvested, counted (A), and analyzed by flow cytometry for expression of CD11b and GR1 (B) as well as DiI-acLDL uptake (C,D).

FIG. 9 shows that Gal-3BP induces a unique macrophage phenotype. Human blood monocytes were incubated with M-CSF (100 ng/ml) for 6 days to produce monocyte-derived macrophages (M0). These macrophages were incubated with interferon-γ (250 units/ml) for 1 day to produce M1 macrophages or with IL-4 (20 units/ml) to produce M2 macrophages or with PGE-2 (1 μM) to produce Mreg macrophages and then challenged with and LPS (10 ng/ml) for another day or with Gal-3BP (10 μg/ml over 48 hours). Only Gal-3BP induced production of IL-2, no IL-12 (typical M1) and moderate IL-10 (typical M2 and Mreg). ** significant, *** highly significant

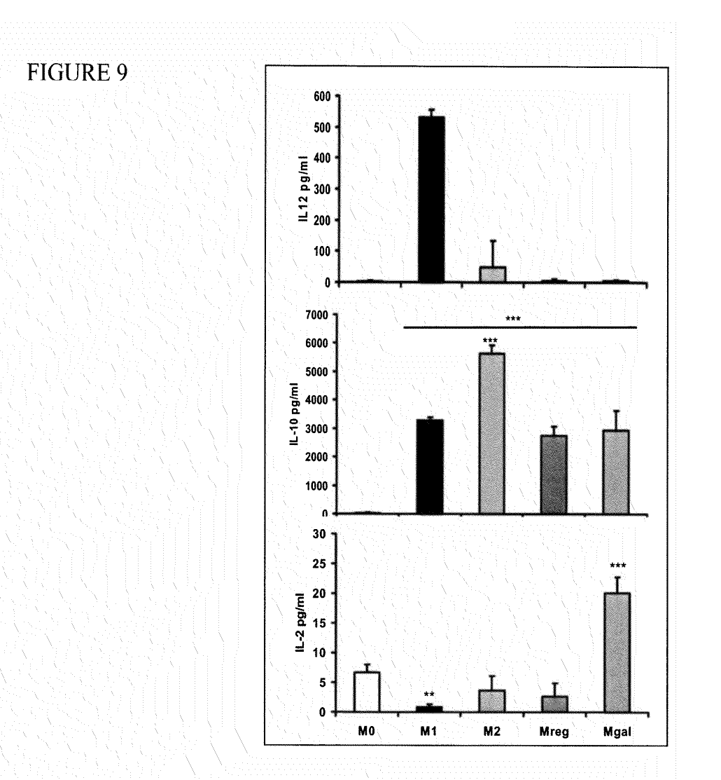

FIG. 10 shows that M1 macrophages produce Gal-3BP. Human blood monocytes were incubated with M-CSF (100 ng/ml) for 6 days to produce monocyte-derived macrophages (M0). These macrophages were incubated with interferon-γ (250 units/ml) for 1 day to produce M1 macrophages or with IL-4 (20 units/ml) to produce M2 macrophages or with PGE-2 (1 μM) to produce Mreg macrophages and then challenged with and LPS (10 ng/ml). Only M1 macrophages secrete significant amounts of Gal-3BP, but levels in supernatant remain about 10 times lower than in blood.

FIGS. 11A-11B show CD36 expression (A) and oxLDL uptake (B) on human monocyte-derived macrophages treated as indicated: untreated (green), isotype control (yellow), Gal3-BP treated (blue), and heat-inactivated Gal3-BP treated (red).

### DETAILED DESCRIPTION

The invention is based, at least in part, on the identification of Galectin-3 binding protein (Gal3-BP, also known as BTBD17B, Mac-2 binding protein or 90K, gene name LGALS3BP in human, also known as CyCAP, MAC-2BP or Ppicap, murin gene name Lgals3bp) as a modulator of atherosclerotic cardiovascular disease. In particular, for example, high levels of Gal-3BP reduce macrophage accumulation and uptake of modified LDL in a dose-dependent manner. Gal-3BP therefore affects both plaque composition and stability. Gal-3BP also inhibits foam cell formation in patients with angiographically confirmed obstructive CAD, and therefore also inhibits formation of cells that contribute to atherosclerotic lesions and development of atherosclerosis.

The invention is also based, at least in part, on the identification of Gal-3BP as a biomarker. In particular, for example, Gal-3BP plasma levels correlate as a positive predictor of improved outcome in coronary artery disease. Low levels of Gal-3BP are therefore indicative of a negative prognosis for coronary artery disease.

Gal-3BP is a secreted 585 (murine 577) amino acid protein and member of the macrophage scavenger receptor cysteine-rich domain superfamily (Koths et al., J. Biol. Chem. 268: 14245 (1993)). Gal-3BP is ubiquitously expressed (Koths et al., J. Biol. Chem. 268: 14245 (1993); and Ullrich et al., J. Biol. Chem. 269: 18401 (1994)) and can be detected in many body fluids like semen, saliva, urine, tears (Koths et al., J. Biol. Chem. 268: 14245 (1993)), milk (D'Ostilio et al., Clin. Exp. Immunol. 104: 543 (1996); and Formarini et al., Clin. Exp. Immunol. 115: 91 (1999)) and plasma, where it is associated with microparticles (Smalley et al., Thromb. Haemost. 97: 67 (2007)).

In two genomic and proteomic screens, Galectin-3 binding protein (Gal-3BP) was expressed in macrophage-derived foam cells and blood microparticles. Gal-3BP dose-dependently downregulates the scavenger receptors CD36 and SR-A at the mRNA and protein levels, leading to decreased uptake of modified LDL (Ac-LDL and ox-LDL). Although Gal-3BP is elevated in patients with coronary artery disease, it is not a risk factor and instead is apparently protective. These findings are relevant to immunologists as a modulator of macrophage phenotype, vascular biologists for a role in atherosclerosis, cardiologists as a biomarker for protection and other clinical investigators, due to a role in inflammatory diseases.

Gal-3BP inhibits foam cell formation through downregulation of CD36 and SR-A resulting in decreased uptake of modified LDL in primary human macrophages. Recombinant murine Gal-3BP reduced oxLDL uptake in peritoneal macrophages. Conversely, bone-marrow derived macrophages from Gal-3BP knockout mice expressed higher levels of CD36, resulting in higher levels of oxLDL in aortic macrophages. In a cohort of 77 patients with angiographically confirmed coronary artery disease, major adverse cardiovascular events were significantly lower in patients with high Gal-313P plasma levels. These findings establish Gal-3BP as a significant modulator of macrophage differentiation and biologically relevant inhibitor of foam cell formation, and Gal-3BP plasma levels can be used to identify coronary artery disease patients at high and low risk for adverse cardiovascular events.

In accordance with the invention, there are provided compositions including Gal-3BP polypeptide. In one embodiment, a composition includes a Gal-313P polypeptide and a pharmaceutically acceptable carrier (e.g., a pharmaceutical composition or formulation, such as saline).

A “polypeptide” refers to two, or more, amino acids linked by an amide or equivalent bond. A polypeptide can also be referred to herein, inter alia, as a protein, peptide, or an amino acid sequence. Polypeptides include at least two, or more, amino acids bound by an amide bond. Polypeptides can form intra or intermolecular disulfide bonds. Polypeptides can also form higher order multimers or oligomers with the same or different polypeptide, or other molecules.

As used herein, the term Gal-3BP polypeptide refers to full length polypeptide sequence, as well as subsequences, fragments or portions of Gal-3BP polypeptide, unless the context indicates otherwise. A subsequence, fragment or portion of Gal-3BP polypeptide means less than the full length reference sequence, which is typically a native full length Gal-3BP polypeptide sequence.

Gal-3BP polypeptide sequences and subsequences include modified forms. In particular embodiments, a modified form retains, at least a part of, a function or activity of an unmodified or reference protein. A “functional polypeptide” or “active polypeptide” refers to a modified polypeptide or a subsequence thereof, such as a Gal-3BP polypeptide or a subsequence thereof, that possesses at least one partial function or biological activity characteristic of a native wild type or full length counterpart polypeptide, for example, Gal-3BP, as disclosed herein, which can be identified through an assay. As disclosed herein, particular non-limiting examples of a function or activity of Gal-3BP polypeptide is to inhibit, reduce, decrease or suppress foam cell formation, macrophage cell expression of scavenger receptor A and/or CD36, macrophage accumulation or uptake of modified LDL (e.g., ox LDL or otherwise modified LDL), plaque formation and formation of atherosclerotic lesions, development of atherosclerosis, and increase, promote or induce secretion of IL-2, etc.

Modified Gal-3BP polypeptide sequences and subsequences of the invention may have an activity or function greater or less than 2-5,5-10, 10-100, 100-1000 or 1000-10.000-fold activity or function than a comparison Gal-3BP polypeptide sequence or subsequence, e.g., to inhibit, reduce, decrease or suppress foam cell formation, macrophage cell expression of scavenger receptor A, expression of CD36, macrophage accumulation, uptake of modified LDL (e.g., ox LDL or otherwise modified LDL), plaque formation and formation of atherosclerotic lesions, development of atherosclerosis, and increase, promote or induce secretion of IL-2, etc.

The invention therefore includes modified forms of Gal-3BP polypeptide sequences and subsequences. Such modified forms typically retain, at least a part of, one or more functions or activities of an unmodified or reference Gal-3BP polypeptide sequence or subsequence.

As used herein, the term “modify” and grammatical variations thereof, means that the composition deviates from a reference composition. Modifications include, for example, substitutions, additions, insertions and deletions to the amino acid sequences set forth herein, which can be referred to as “variants.” Exemplary sequence substitutions, additions, and insertions include a full length or a portion of a sequence with one or more amino acids substituted, added or inserted, for example of Gal-3BP polypeptide sequence, wherein the modified Gal-3BP polypeptide inhibits, reduces, decreases or suppresses foam cell formation, macrophage cell expression of scavenger receptor A and/or CD36, macrophage accumulation or uptake of modified LDL (e.g., ox LDL or otherwise modified LDL), plaque formation and formation of atherosclerotic lesions, development of atherosclerosis, or increases, promotes or induces secretion of IL-2, etc.

Modified polypeptides include, for example, non-conservative and conservative substitutions of Gal-3BP polypeptide sequences. In particular embodiments, a modified protein has one or a few (e.g., 1-5%, 5-10%, 10-20% or 20-30% of the residues of total protein length, or 2-10, 10-20, 20-30, 30-40, 40-50, 50-60, 60-75, 75-100 residues, substituted) conservative or non-conservative substitutions.

As used herein, the term “conservative substitution” denotes the replacement of an amino acid residue by another, chemically or biologically similar residue. Biologically similar means that the substitution does not destroy a biological activity or function. Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carbobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide. Further modified forms include sugars, or glycosylated proteins.

Modified forms of protein (e.g., Gal-3BP polypeptide sequence or subsequence), and other compositions, include additions and insertions. For example, an addition can be the covalent or non-covalent attachment of any type of molecule to a protein (e.g., Gal-3BP) or other composition. Typically additions and insertions confer a distinct function or activity.

Additions and insertions include fusion polypeptide sequence constructs, which is a sequence (e.g., Gal-3BP) having one or more molecules not normally present in a reference native (wild type) sequence (e.g., Gal-3BP) covalently attached to the sequence. A particular example is an amino acid sequence of another protein (e.g., immunoglobulin such as an Fc domain, or antibody) attached to produce a chimeric polypeptide to impart a distinct function (e.g., increased solubility, in vivo half life, etc.).

Additional non-limiting examples of amino acid modifications include protein subsequences and fragments. Exemplary Gal-3BP subsequences and fragments include a Gal-3BP polypeptide fragment or a portion of that inhibits, reduces, decreases or suppresses foam cell formation, macrophage cell expression of scavenger receptor A and/or CD36, macrophage accumulation or uptake of modified LDL (e.g., ox LDL or otherwise modified LDL), plaque formation and formation of atherosclerotic lesions, development of atherosclerosis, and increase, promote or induce secretion of IL-2, etc.

Non-limiting subsequences of full length Galectin-3 binding protein (Gal-3BP) include amino acids having a length of about 5-10, 10-20, 20-25, 25-50, 50-100, 100-150, 150-200, 200-250, 250-300, 300-350, 350-400, 400-500, 500-600 or more amino acids in length, and less than full length Gal-3BP polypeptide sequence, e.g., a native (naturally occurring) sequence. Gal-3BP subsequences, fragments and portions can retain all or a part of a function or activity of full length Gal-3BP polypeptide (e.g., inhibit, reduce, decrease or suppress foam cell formation, macrophage cell expression of scavenger receptor A and/or CD36, macrophage accumulation or uptake of modified LDL (e.g., ox LDL or otherwise modified LDL), plaque formation and formation of atherosclerotic lesions, development of atherosclerosis, and increase, promote or induce secretion of IL-2, etc.).

Studies set forth herein disclose several Gal-3BP polypeptide subsequences, fragments and portions that retain all or a part of a function or activity of full length Gal-3BP polypeptide. In particular embodiments, Gal-3BP polypeptide subsequences, fragments and portions include residues 24-124 (SRCR domain); residues 153-221 (BTB domain); and residues 260-360 (BACK domain) of Gal-3BP polypeptide. In additional particular embodiments, Gal-3BP polypeptide subsequences, fragments and portions include subsequences of residues 24-124 (SRCR domain), e.g., residues 25-120 of SRCR domain; residues 153-221 (BTB domain), e.g., residues 155-218 of BTB domain; and residues 260-360 (BACK domain), e.g., residues 262-357 of BACK domain.

Gal-3BP polypeptide also refers to polypeptide sequences having sequence identity to a reference Gal-3BP polypeptide sequence. Such Gal-3BP polypeptide sequences can have at least about 60%, 65%, 70%, 75%, 80%, 85%, 90%, 95%, 96%, 97%, 98%, 99% or more identity (homology) to a reference Gal-3BP polypeptide sequence (e.g., a mammalian Gal-3BP polypeptide sequence, such as human Gal-3BP polypeptide sequence). Such Gal-3BP polypeptide sequences with at least about 60%, 65%, 70%, 75%, 80%, 85%, 90%, 95%, 96%, 97%, 98%, 99% or more identity (homology) to a reference Gal-3BP polypeptide sequence can have sufficient identity to retain all or a part of a function or activity of a reference Gal-3BP polypeptide (e.g., inhibit, reduce, decrease or suppress foam cell formation, macrophage cell expression of scavenger receptor A and/or CD36, macrophage accumulation or uptake of modified LDL (e.g., ox LDL or otherwise modified LDL), plaque formation and formation of atherosclerotic lesions, development of atherosclerosis, and increase, promote or induce secretion of IL-2, etc.).

The term “identity” and grammatical variations thereof, mean that two or more referenced entities are the same. Thus, where two polypeptide sequences (e.g., Gal-3BP polypeptide sequences) are identical, they have the same amino acid sequence, at least within the referenced region or portion. Where two nucleic acid sequences are identical, they have the same polynucleotide sequence, at least within the referenced region or portion. The identity can be over a defined area (region or domain) of the sequence. An “area of identity” refers to a portion of two or more referenced entities that are the same. Thus, where two protein or nucleic acid sequences are identical over one or more sequence regions they share identity within that region.

The percent identity can extend over the entire sequence length of the polypeptide (e.g., a Gal-3BP polypeptide sequence). In particular aspects, the length of the sequence sharing the percent identity is 5 or more contiguous amino acids, e.g., 5, 6, 7, 8, 9, 10, 11, 12, 13, 14, 15, 16, 17, 18, 19, 20, 21, 22, 23, 24, 25, etc. contiguous amino acids. In additional particular aspects, the length of the sequence sharing the percent identity is 25 or more contiguous amino acids, e.g., 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet additional particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The extent of identity (homology) between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch -2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol. Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

An exemplary full length human Gal-3BP polypeptide sequence (SEQ ID NO:1) is as follows:

```
                10 20 30 40 50 60
MTPPRLFWVW LLVAGTQGVN DGDMRLADGG ATNQGRVEIF YRGQWGTVCD NLWDLTDASV
            70 80 90 100 110 120
VCRALGFENA TQALGRAAFG QGSGPIMLDE VQCTGTEASL ADCKSLGWLK SNCRHERDAG
              130 140 150 160 170 180
VVCTNETRST HTLDLSRELS EALGQIFDSQ RGCDLSISVN VQGEDALGFC GHTVILTANL
              190 200 210 220 230 240
EAQALWKEPG SNVTMSVDAE CVPMVRDLLR YFYSRRIDIT LSSVKCFHKL ASAYGARQLQ
              250 260 270 280 290 300
GYCASLFAIL LPQDPSFQMP LDLYAYAVAT GDALLEKLCL QFLAWNFEAL TQAEAWPSVP
              310 320 330 340 350 360
TDLLQLLLPR SDLAVPSELA LLKAVDTWSW GERASHEEVE GLVEKIRFPM MLPEELFELQ
              370 380 390 400 410 420
FNLSLYWSHE ALFQKKTLQA LEFHTVPFQL LARYKGLNLT EDTYKPRIYT SPTWSAFVTD
              430 440 450 460 470 480
SSWSARKSQL VYQSRRGPLV KYSSDYFQAP SDYRYYPYQS FQTPQHPSFL FQDKRVSWSL
              490 500 510 520 530 540
VYLPTIQSCW NYGFSCSSDE LPVLGLTKSG GSDRTIAYEN KALMLCEGLF VADVTDFEGW
              550 560 570 580
KAAIPSALDT NSSKSTSSFP CPAGHFNGFR TVIRPFYLTN SSGVD
```

Additional representative mammalian (human, Pan troglodytes, Canis lupus familiaris, Bos Taurus, Mus musculus and Rattus norvegicus) sequences (SEQ ID NOs:1-6), and an alignment showing the regions of identity, are illustrated as follows:

| | | | |
|---|---|---|---|
| NP_005558.1 | 1 | -----------------------MTPPRLFWVWLLVA-----------GT | 16 |
| XP_001158328.1 | 1 | -----------------------MTPPRLFWVWLLVA-----------GT | 16 |
| XP_540464.2 | 1 | -----------------------MALPLVLWMCLLVA-----------GT | 16 |
| NP_001039781.1 | 1 | -----------------------MAPLRLFWIWLLVV-----------GT | 16 |
| NP_035280.1 | 1 | -----------------------MALLWLLSVFLLVP-----------GT | 16 |
| NP_620796.1 | 1 | -----------------------MALLWLLSVFLLVP-----------GT | 16 |
| NP_005558.1 | 17 | QGVNDGDMRLADGGATNQGRVEIFYRGQWGTVCDNLWDLTDASVVCRALG | 66 |
| XP_001158328.1 | 17 | QGVNDGDMRLADGGATNQGRVEIFYRGQWGTVCDNLWDLTDASVVCRALG | 66 |
| XP_540464.2 | 17 | QGVKDGDMRLANGDTANEGRVEIFYSGRWGTVCDNLWDLMDASVVCRALG | 66 |
| NP_001039781.1 | 17 | RGVKDGDMRLADGGSANQGRVEIYYNGQWGTVCENMWDLTDASVVCRALG | 66 |
| NP_035280.1 | 17 | QGTEDGDMRLVNGASANEGRVEIFYRGRWGTVCDNLWNLLDAHVVCRALG | 66 |
| NP_620796.1 | 17 | QGAKDGDMRLVNGASASEGRVEIFYRGRWGTVCDNLWNLLDAHVVCRALG | 66 |
| NP_005558.1 | 67 | FENATQALGRAAFGQGSGPIMLDEVQCTGTEASLADCKSLGWLKSNCRHE | 116 |
| XP_001158328.1 | 67 | FENATQALGRAAFGQGSGPIMLDEVQCMGTEASLADCKSLGWLKSNCRHE | 116 |
| XP_540464.2 | 67 | FENATEALGGAAFGPGKGPIMLDEVECTGTEPSLANCTSLGWMKSNCRHN | 116 |
| NP_001039781.1 | 67 | FQNATEALGGAAFGPGYGPIMLDEVRCTGTEPSLANCSSLGWMRSNCRHD | 116 |
| NP_035280.1 | 67 | YENATQALGRAAFGPGKGPIMLDEVECTGTESSLASCRSLGWMVSRCGHE | 116 |
| NP_620796.1 | 67 | YENATQALSRAAFGPGKGPIMLDEVECTGNESSLANCSSLGWMVSHCGHE | 116 |
| NP_005558.1 | 117 | RDAGVVCTNETRSTHTLDLSRE----LSEALGQIFDSQRGCDLSISVN-V | 161 |
| XP_001158328.1 | 117 | RDAGVVCTNETRSTHTLDLSRE----LSEALGQIFDSQRGCDLSISVN-V | 161 |
| XP_540464.2 | 117 | QDAGVVCSNETRGAHTLDLSGE----LPAALEQIFDSQRGCDLSIRVK-V | 161 |
| NP_001039781.1 | 117 | KDASVICTNETRGVYTLDLSGE----LPAALEQIFESQKGCDLFITVK-V | 161 |
| NP_035280.1 | 117 | KDAGVVCSNDTTGLHILDLSGE----LSDALGQIFDSQQGCDLFIQVT-G | 161 |
| NP_620796.1 | 117 | KDAGVVCSNDSRGIHILDLSGE----LPDALGQIFDSQQDCDLFIQVT-G | 161 |
| NP_005558.1 | 162 | QGEDALG--FCGHTVILTANLEAQALWKEPGSNVTMSVDAECVPMVRDLL | 209 |
| XP_001158328.1 | 162 | QGEDALG--FCGHTVILTANLEAQALWKEPGSNVTMSVDAECVPMVRDLL | 209 |
| XP_540464.2 | 162 | KDQEEEGPHFCAHRLILAANPEAQALCKAPGSTVTMEVDAECLPVVRDFI | 211 |
| NP_001039781.1 | 162 | REEDEIA--MCAHKLILSTNPEAHGLWKEPGSRVTMEVDAECVPVVKDFI | 209 |
| NP_035280.1 | 162 | QGYEDLS--LCAHTLILRTNPEAQALWQVVGSSVIMRVDAECMPVVRDFL | 209 |
| NP_620796.1 | 162 | QGHGDLS--LCAHTLILRTNPEAQALWQVVGSSVIMRVDAECMPVVRDFL | 209 |
| NP_005558.1 | 210 | RYFYSRRIDITLSSVKCFHKLASAYGARQLQGYCASLFAILLPQDPSFQM | 259 |
| XP_001158328.1 | 210 | RYFYSRRIDITLSSVKCFHKLASAYGARQLQGYCASLFAILLPRDPSFQT | 259 |
| XP_540464.2 | 212 | RYLYSRRLDISLTSVKCFHKLASAYEAQQLQSFCASLFAILLPEDPSFQA | 261 |
| NP_001039781.1 | 210 | RYLYSRRIDVSLSSVKCLHKFASAYQAKQLQSYCGHLFAILIPQDPSFWT | 259 |
| NP_035280.1 | 210 | RYFYSRRIEVSMSSVKCLHKLASAYGATELQDYCGRLFATLLPQDPTFHT | 259 |
| NP_620796.1 | 210 | RYFYSRRIEVSMSSVKCLHKLASAYGATELQGYCGRLFVTLLPQDPTFHT | 259 |
| NP_005558.1 | 260 | PLDLYAYAVATGDALLEKLCLQFLAWNFEALTQAEAWPSVPTDLLQLLLP | 309 |
| XP_001158328.1 | 260 | PLDLYAYAVATGDALLEKLCLQFLAWNFEALTQAEAWPSVPTDLLQLLLP | 309 |
| XP_540464.2 | 262 | PLDLYAYALATQDPVLEELCVQFLAWNFEGLTQATAWPRVPTALLQLLLS | 311 |
| NP_001039781.1 | 260 | PLELYAYALATRDTVLEEICVQFLAWNFGALTQAEAWPSVPPALLQGLLS | 309 |
| NP_035280.1 | 260 | PLDLYAYARATGDSMLEDLCVQFLAWNFEPLTQSESWSAVPTTLIQALLP | 309 |
| NP_620796.1 | 260 | PLELYEYAQATGDSVLEDLCVQFLAWNFEPLTQAEAWLSVPNALIQALLP | 309 |
| NP_005558.1 | 310 | RSDLAVPSELALLKAVDTWSWGERA--SHEEVEGLVEKIRFPMMLPEELF | 357 |
| XP_001158328.1 | 310 | RSDLAVPSELALLKAVDTWSWGERA--SHEEVEDLVEKIRFPMMLPEELF | 357 |
| XP_540464.2 | 312 | RSELAVPSELALLTALDVWSQERRP--SHGEVARLVDKVRFPNMLPEHLF | 359 |
| NP_001039781.1 | 310 | RTELVVPSELVLLLAVDKWSQERHT--SHKEVEALVGQVRFPMKPPQDLF | 357 |
| NP_035280.1 | 310 | KSELAVSSELDLLKAVDQWSTETIA--SHEDIERLVEQVRFPMMLPQELF | 357 |
| NP_620796.1 | 310 | KSELAVSSELDLLKAVDQWSTATGA--SHGDVERLVEQIRFPMMLPQELF | 357 |
| NP_005558.1 | 358 | ELQFNLS-LYWSHEALFQKKTLQALEFHTVPFQLLARYKGLNLTEDTYKP | 406 |
| XP_001158328.1 | 358 | ELQFNLS-LYWSHEALFQKKTLQALEFHTVPFQLLARYKGLNLTEDTYKP | 406 |
| XP_540464.2 | 360 | ELQFNLS-LYWSHEALFQKKILQALEFHTVPFRLLAQHRGLNLTEDAYQP | 408 |
| NP_001039781.1 | 358 | SLQFNLS-LYWSHEALFQKKILQALEFHTVPFELLAQYWGLNLTEGTYQP | 406 |
| NP_035280.1 | 358 | ELQFNLS-LYQDHQALFQRKTMQALEFHTVPVEVLAKYKGLNLTEDTYKP | 406 |
| NP_620796.1 | 358 | ELQFNLS-LYQGHQALFQRKTMEALEFHTVPLKVLAKYRSLNLTEDVYKP | 406 |
| NP_005558.1 | 407 | RIYTSPTWSAFVTDSSWSARKSQLVYQSRRGPLVKYSSDYFQAPSDYRYY | 456 |
| XP_001158328.1 | 407 | RIYTSPTWSASVTDSSWSARKSQLVYQSRRGPLVKYSSNYFQAPSDYRYY | 456 |
| XP_540464.2 | 409 | RLYTSPTWSASVSRSS----------------------------SRYWNY | 430 |
| NP_001039781.1 | 407 | RLYTSPTWSQSVMSSS--------------------------------YN | 424 |
| NP_035280.1 | 407 | RLYTSSTWSSLVMASTWRAQRYEYNRYNQ--------LYTYGYGSVARYN | 448 |
| NP_620796.1 | 407 | RLYTSSTWSSLLMAGAWSTQSY---KYRQ--------FYTYNYGSQSRYS | 445 |
| NP_005558.1 | 457 | PYQSF-QTPQHPSFLFQDKRVSWSLVYLPTIQSCWNYGFSCSSDELPVLG | 505 |
| XP_001158328.1 | 457 | PYQSF-QTPQHPSFLFQDKRVSWSLVYLPTIQSCWNYGFSCSSDELPVLG | 505 |
| XP_540464.2 | 431 | PYQSF-QTPQMPSFLFQNKYISWSLVYLPTVQSCWNYGFSCSSDEVPLLG | 479 |
| NP_001039781.1 | 425 | PSRSF-QTPQHPSFLFHDSSVSWSFVYLPTLQSCWNYGFSCSSDDPPLLA | 473 |
| NP_035280.1 | 449 | SYQSF-QTPQHPSFLFKDKQISWSATYLPTMQSCWNYGFSCTSNELPVLG | 497 |
| NP_620796.1 | 446 | SYQNF-QTPQHPSFLFKDKLISWSATYLPTIQSCWNYGFSCTSDELPVLG | 494 |
| NP_005558.1 | 506 | LTKSGG--SDRTIAYENKALMLCEGL-FVADVTDFEGWKAAIPSALDTNS | 552 |
| XP_001158328.1 | 506 | LTKSGG--SDRTIAYENKALMLCEGL-FVADVTDFEGWKAAIPSALDINS | 552 |
| XP_540464.2 | 480 | LSKSDY--SDPTIGYENKALMRCGGR-FVADVTDFEGQKALIPSALGTNS | 526 |
| NP_001039781.1 | 474 | LSKSSYSKSNPTIGYENRALLHCEGS-FVVDVIDFKGWKALVPSALATNS | 522 |
| NP_035280.1 | 498 | LTTSSY--SNPTIGYENRVLILCGGY-SVVDVTSFEGSKAPIPTALDTNS | 544 |
| NP_620796.1 | 495 | LTTSSY--SDPTIGYENKALILCGGY-SVVDVTTFIGSKAPIPGTQETNS | 541 |
| NP_005558.1 | 553 | SKSTSSFPCPAGHFNGFRTVIRPFYLTNSSGVD | 585 |
| XP_001158328.1 | 553 | SKSTSSFPCPAGHFNGFRTVIRPFYLTNSSGVD | 585 |
| XP_540464.2 | 527 | SRRPSLFPCLGGSFSSFQVVIRPFYLTNSSDVD | 559 |
| NP_001039781.1 | 523 | SRSTSLFPCPSGVFSRFQVVIRPFYLTNSTDMD | 555 |
| NP_035280.1 | 545 | SKTPSLFPCASGAFSSFRVVIRPFYLTNSTDMV | 577 |
| NP_620796.1 | 542 | SKTPSLFPCASGAFSSFREVIRPFYLTNSTDTE | 574 |

| | | | % Identity vs. Homo |
|---|---|---|---|
| Protein Acc. | Gene | Organism | sapiens (protein) |
| NP_005558.1 | LGALS3BP | Homo sapiens | |
| XP_001158328.1 | LGALS3BP | Pan troglodytes | 98.8 |
| XP_540464.2 | LGALS3BP | Canis lupus familiaris | 76.1 |
| NP_001039781.1 | LGALS3BP | Bos taurus | 72.0 |
| NP_035280.1 | Lgals3bp | Mus musculus | 69.8 |
| NP_620796.1 | Lgals3bp | Rattus norvegicus | 68.1 |

Modifications can be produced using methods known in the art (e.g., PCR based site-directed, deletion and insertion mutagenesis, chemical modification and mutagenesis, cross-linking, etc.), or may be spontaneous or naturally occurring (e.g. random mutagenesis). For example, naturally occurring Gal-3BP polypeptide sequence allelic variants can occur by alternative RNA splicing, polymorphisms, or spontaneous mutations of a nucleic acid encoding Gal-3BP polypeptide. Further, deletion of one or more amino acids can also result in a modification of the structure of the resultant polypeptide without significantly altering a biological function or activity. Deletion of amino acids can lead to a smaller active molecule. For example, as set forth herein, removal of certain Gal-3BP polypeptide amino acids does not destroy the ability to inhibit, reduce, decrease or suppress foam cell formation, macrophage cell expression of scavenger receptor A and/or CD36, macrophage accumulation or uptake of modified LDL (e.g., ox LDL or otherwise modified LDL), plaque formation and formation of atherosclerotic lesions, development of atherosclerosis, and increase, promote or induce secretion of IL-2, etc.).

In accordance with the invention, there are provided isolated and purified Gal-3BP polypeptides, as well as modified forms, such as subsequences, fragments and portions of Gal-3BP polypeptides. The term “isolated,” when used as a modifier of a composition (e.g., Gal-3BP polypeptide sequences, subsequences, modified forms, nucleic acids encoding same, antibodies, etc.), means that the compositions are made by the hand of man or are separated, completely or at least in part, from their naturally occurring in vivo environment. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., a Gal-3BP polypeptide sequence) can also be “substantially pure” or “purified” when free of most or all of the materials with which it typically associates with in nature. Generally, isolated compositions are substantially free of one or more materials with which they normally associate with in nature, for example, one or more protein, nucleic acid, lipid, carbohydrate, cell membrane. Thus, an isolated sequence that also is substantially pure or purified does not include polypeptides or polynucleotides present among millions of other sequences, such as antibodies of an antibody library or nucleic acids in a genomic or cDNA library, for example. Typically, purity can be at least about 50%, 60% or more by mass. The purity can also be about 70% or 80% or more, and can be greater, for example, 90% or more. Purity can be determined by any appropriate method, including, for example, UV spectroscopy, chromatography (e.g., HPLC, gas phase), gel electrophoresis and sequence analysis (nucleic acid and peptide), and is typically relative to the amount of impurities, which typically does not include inert substances, such as water.

A “substantially pure” or “purified” composition can be combined with one or more other molecules. Thus, “substantially pure” or “purified” does not exclude combinations of compositions, such as combinations of Gal-3BP polypeptide sequences, subsequences, antibodies, and other antibodies, agents, drugs or therapies.

As used herein, the term “recombinant,” when used as a modifier of polypeptides, polynucleotides and antibodies, means that the compositions have been manipulated (i.e., engineered) in a fashion that generally does not occur in nature (e.g., in vitro). A particular example of a recombinant polypeptide would be where a Gal-3BP polypeptide is expressed by a cell transfected with a polynucleotide encoding the Gal-3BP polypeptide. A particular example of a recombinant polynucleotide would be where a nucleic acid (e.g., genomic or cDNA) encoding Gal-3BP polypeptide cloned into a plasmid, with or without 5′, 3′ or intron regions that the gene is normally contiguous with in the genome of the organism. Another example of a recombinant polynucleotide or polypeptide is a hybrid or fusion sequence, such as a chimeric Gal-3BP polypeptide sequence comprising and a second sequence, such as a heterologous functional domain.

Compositions including Gal-3BP polypeptide can include any amount or dose of Gal-3BP polypeptide. In particular embodiments, Gal-3BP polypeptide is in a concentration range of about 10 μg/ml to 100 mg/ml, or in a range of about 100 μg/ml to 10 mg/ml, or at a concentration of about 1 mg/ml. In further particular embodiments, Gal-3BP polypeptide is in an amount of 10-100 milligrams, or an amount of 10-50 milligrams.

Compositions, including Gal-3BPpolypeptides, subsequences and modified forms as disclosed herein, as well as modified or unmodified full length native (e.g., wild-type) Gal-3BP polypeptide sequences (e.g., mammalian, such as human Gal-3BP polypeptide sequences), are useful in various treatment, diagnostic, detection, screening and use methods. Compositions and methods of the invention are applicable to treating numerous disorders and diseases, both chronic and acute. Disorders and diseases treatable in accordance with the invention include, but are not limited to, treatment of an acute and chronic adverse cardiovascular events and cardiovascular diseases, such as coronary artery disease, peripheral artery disease, cerebrovascular disease, renal artery disease, stroke, myocardial infarction (heart attack), ischemic heart failure, transient ischemic attack or brain trauma. Disorders treatable in accordance with the invention also include, but are not limited to treatment of an acute and chronic artherosclerotic plaque formation, for example, reducing or decreasing risk of artherosclerotic plaque formation, and reducing or inhibiting artherosclerotic plaque formation.

In accordance with the invention, there are provided methods of treating an acute and chronic disorders and diseases in which a subject would benefit from a Gal-3BP polypeptide sequence. In one embodiment, a method includes administering a Gal-3BP polypeptide sequence to a subject (e.g., having or at risk of an adverse cardiovascular event) in an amount effective to reduce or decrease risk of the adverse cardiovascular event or cardiovascular disease in the subject. In particular aspects, a subject has or is at risk of having coronary artery disease, peripheral artery disease, cerebrovascular disease, renal artery disease, stroke, myocardial infarction (heart attack), ischemic heart failure, transient ischemic attack or brain trauma.

In another embodiment, a method includes administering a Gal-3BP polypeptide sequence to a subject (e.g., having or at risk of an artherosclerotic plaque formation) in an amount effective to decrease risk of artherosclerotic plaque formation in the subject. In an additional embodiment, a method includes administering a Gal-3BP polypeptide sequence to a subject (e.g., having or at risk of an artherosclerotic plaque formation) in an amount effective to reduce or inhibit artherosclerotic plaque formation in the subject.

In a further embodiment, a method includes administering a Gal-3BP polypeptide sequence to a subject (e.g., having undesirable foam cells or at risk of undesirable foam cell formation) in an amount effective to decrease foam cells or the risk of foam cell formation in the subject.

In various embodiments of the invention, a method results in increasing the amount Gal-3BP polypeptide sequence in the subject, thereby effecting treatment of the subject. Amounts may vary depending upon the subject, the desired effect, and the disorder or disease, or risk of disorder or disease, to be treated. Amounts of Gal-3BP polypeptide can be reflected in blood or plasma. In particular aspects, Gal-3BP polypeptide sequence increases to an amount in the subject of greater than 2 ug/ml in blood plasma, increases to an amount in the subject of greater than 5 ug/ml in blood plasma, increases to an amount in the subject of greater than 10 ug/ml in blood plasma, increases to an amount in the subject of greater than 15 ug/ml in blood plasma, or increases to an amount in the subject of greater than 20 ug/ml in blood plasma. Increased amounts of Gal-3BP polypeptide sequence may be transient, or longer term (e.g., minutes, hours, days, weeks, etc.). In particular aspects, Gal-3BP polypeptide sequence increases to an amount for a period of time greater than 12, 24, 36, 48, 72 hours, or at least 3, 4, 5, 6, 7, 8, 9, 10, 12, 14 days, weeks or months.

The term “contacting” means direct or indirect binding or interaction between two or more entities (e.g., between a Gal-3BP polypeptide sequence and a target). A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration, or delivery.

In methods of the invention, a compound can be administered prior to, substantially contemporaneously with or following an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation or foam cells or foam cell formation), or one or more adverse symptoms, disorders, illnesses, pathologies, diseases, or complications caused by or associated with, for example, an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation or foam cells or foam cell formation). Thus, methods of the invention may be practiced prior to (i.e. prophylaxis), concurrently with or after evidence of the disorder or disease begins (e.g., one or more symptoms of an adverse cardiovascular event, artherosclerotic plaque formation, or foam cell formation), or one or more adverse symptoms, disorders, illnesses, pathologies, diseases, or complications caused by or associated with an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation or foam cells or foam cell formation). Administering a composition prior to, concurrently with or immediately following development of a symptom may decrease, reduce, inhibit, suppress, limit or control the occurrence, frequency, severity, progression, or duration of one or more adverse symptoms, disorders, illnesses, pathologies, diseases, or complications caused by or associated with the adverse cardiovascular event or cardiovascular disease in the subject. In addition, administering a composition prior to, concurrently with or immediately following development of one or more symptoms may decrease, reduce, inhibit, suppress, limit, control or prevent damage to cells, tissues or organs that occurs, for example, due to one or more adverse symptoms, disorders, illnesses, pathologies, diseases, or complications caused by or associated with the adverse cardiovascular event or cardiovascular disease

In methods of the invention, a compound can be administered prior to, substantially contemporaneously with or following administering a second drug or treatment. Non-limiting examples of classes of second drugs or treatments include blood pressure reducing or diabetes medicines, such as ACE inhibitors, calcium antagonists, and beta-blockers, and triglyceride and cholesterol reducing medicines, such as statins.

Compositions and the methods of the invention, such as treatment methods, can provide a detectable or measurable therapeutic benefit or improvement to a subject. A therapeutic benefit or improvement is any measurable or detectable, objective or subjective, transient, temporary, or longer-term benefit to the subject or improvement in the disorder or disease, or one or more adverse symptoms, disorders, illnesses, pathologies, diseases, or complications caused by or associated with the disorder or disease. Therapeutic benefits and improvements include, but are not limited to, decreasing, reducing, inhibiting, suppressing, limiting or controlling the occurrence, frequency, severity, progression, or duration of an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, or complications caused by or associated with an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, or one or more adverse symptoms, disorders, illnesses, pathologies, diseases, or complications caused by or associated with an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation or foam cells or foam cell formation. Compositions and methods of the invention therefore include providing a therapeutic benefit or improvement to a subject.

In the methods of the invention in which a therapeutic benefit or improvement is a desired outcome, a composition of the invention such as a Gal-3BP polypeptide sequence, can be administered in a sufficient or effective amount to a subject in need thereof. An “effective amount” or “sufficient amount” refers to an amount that provides, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured). For example, a sufficient amount of a Gal-3BP polypeptide sequence, is considered as having a therapeutic effect if administration results in a decreased or reduced amount or frequency of therapy for treatment of an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, or one or more adverse symptoms, disorders, illnesses, pathologies, diseases, or complications caused by or associated with an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation or foam cells or foam cell formation.

The doses of an “effective amount” or “sufficient amount” for treatment (e.g., to provide a therapeutic benefit or improvement) typically are effective to ameliorate a disorder or disease, or one, multiple or all adverse symptoms, consequences or complications of the disorder or disease, one or more adverse symptoms, disorders, illnesses, pathologies, diseases, or complications, for example, caused by or associated with an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, to a measurable extent, although decreasing, reducing, inhibiting, suppressing, limiting or controlling a progression or worsening of the disorder or disease, or a symptom thereof, is a satisfactory outcome.

The term “ameliorate” means a detectable improvement in a subject's condition. A detectable improvement includes a subjective or objective decrease, reduction, inhibition, suppression, limit or control in the occurrence, frequency, severity, progression, or duration of the disorder or disease, such as an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, or one or more adverse symptoms, disorders, illnesses, pathologies, diseases, or complications caused by or associated with an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, or an improvement in an underlying cause or a consequence of the disorder or disease e.g., an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation), or a reversal of the disorder or disease (e.g., an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation).

Treatment can therefore result in decreasing, reducing, inhibiting, suppressing, limiting, controlling or preventing a disorder or disease, or an associated symptom or consequence, or underlying cause; decreasing, reducing, inhibiting, suppressing, limiting, controlling or preventing a progression or worsening of a disorder or disease, symptom or consequence, or underlying cause; or further deterioration or occurrence of one or more additional symptoms of the disorder or disease, or symptom. Thus, a successful treatment outcome can lead to a “therapeutic effect,” or “benefit” of decreasing, reducing, inhibiting, suppressing, limiting, controlling or preventing the occurrence, frequency, severity, progression, or duration of an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation, or one or more symptoms or underlying causes or consequences of an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation in the subject. Treatment methods affecting one or more underlying causes of the disorder or disease or symptom are therefore considered to be beneficial. Stabilizing a disorder or disease is also a successful treatment outcome.

A therapeutic benefit or improvement therefore need not be complete ablation of the disorder or disease, or any one, most or all symptoms, complications, consequences or underlying causes associated with the disorder or disease. Thus, a satisfactory endpoint is achieved when there is an incremental improvement in a subject's disorder or disease, or a partial decrease, reduction, inhibition, suppression, limit, control or prevention in the occurrence, frequency, severity, progression, or duration, or inhibition or reversal, of the disorder or disease or one or more associated adverse symptoms or complications or consequences or underlying causes, worsening or progression (e.g., stabilizing one or more symptoms or complications of the disorder or disease), of the disorder or disease, such as an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, or one or more adverse symptoms, disorders, illnesses, pathologies, diseases, or complications caused by or associated with an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation or foam cells or foam cell formation, over a short or long duration of time (hours, days, weeks, months, etc.).

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, status of the disorder, disease or condition treated or the side effects of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered sufficient also include amounts that result in a reduction of the use of another treatment, therapeutic regimen or protocol.

An effective amount or a sufficient amount need not be effective in each and every subject treated, prophylactically or therapeutically, nor a majority of treated subjects in a given group or population. An effective amount or a sufficient amount means effectiveness or sufficiency in a particular subject, not a group or the general population. As is typical for such methods, some subjects will exhibit a greater response, or less or no response to a treatment method.

Particular non-limiting examples of therapeutic benefit or improvement for a disorder or disease include decreasing, reducing, inhibiting, suppressing, limiting, controlling or preventing occurrence, frequency, severity, progression, or duration of one or more adverse cardiovascular events or cardiovascular diseases, such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation. Additional particular non-limiting examples of therapeutic benefit or improvement include stabilizing the disorder or disease (i.e., decreasing, reducing, inhibiting, suppressing, limiting, controlling or preventing a worsening or progression of a symptom or complication associated with an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation). Symptoms or complications associated with an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation whose occurrence, frequency, severity, progression, or duration can be decreased, reduced, inhibited, suppressed, limited, controlled or prevented are known to one of skill in the art. A therapeutic benefit can also include reducing susceptibility of a subject to an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation, or accelerating recovery from one or more adverse symptoms, disorders, illnesses, pathologies, diseases, or complications caused by or associated with an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation.

Effectiveness of a treatment method, such as a therapeutic benefit or improvement for a disorder or disease, can be ascertained by various methods. Such methods include, for example, determining artherosclerotic plaque formation by measuring carotid intima-media thickness (IMT); determining artherosclerotic plaque formation by imaging; determining artherosclerotic plaque formation by CT scanning, MRI, coronary angiography, intravascular ultrasound (IVUS), molecular contrast imaging, or molecular ultrasound contrast imaging; determining artherosclerotic plaque formation by cardiac spiral CT and measuring calcium carbonate deposits.

As is typical for treatment or therapeutic methods, some subjects will exhibit greater or less response to a given treatment, therapeutic regiment or protocol. Thus, appropriate amounts will depend upon the condition treated (e.g., the extent or severity of the atherosclerosis or plaque formation), the therapeutic effect desired, as well as the individual subject (e.g., the bioavailability within the subject, gender, age, etc.).

The term “subject” refers to animals, typically mammalian animals, such as humans, non human primates (apes, gibbons, chimpanzees, orangutans, macaques), domestic animals (dogs and cats), farm animals (horses, cows, goats, sheep, pigs) and experimental animal (mouse, rat, rabbit, guinea pig). Subjects include animal disease models, for example, animal models of adverse cardiovascular events and cardiovascular diseases such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation, etc. or for studying in vivo a composition of the invention, for example, a Gal-3BP polypeptide sequence.

Subjects appropriate for treatment include those that have had, are having or at risk of having an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation. Such subjects include those undergoing treatment for a adverse cardiovascular eventor cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation, as well as those who have had or have undergone treatment or therapy for an adverse cardiovascular event such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation. Specific non-limiting examples include subjects that have had or are at risk for an adverse cardiovascular event or a cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation.

“At risk” subjects typically have increased risk factors for an adverse cardiovascular event or cardiovascular disease such as coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation. Particular subjects at risk include total cholesterol levels, such as levels at or above 100 mg/dL, 150 mg/dL, 200 mg/dL, or 250 mg/dL. Additional particular subjects at risk include Gal-3BP polypeptide levels less than certain amounts, such as Gal-3BP polypeptide less than 10 ug/ml, 9 ug/ml, 8 ug/ml, 7 ug/ml, 6 ug/ml, or less than 5 ug/ml, or less than 3 ug/ml. Further particular subjects at risk include C reactive protein (CRP) levels greater than certain amounts, for example, greater than 1 ug/ml, or 2 ug/ml, or 3 ug/ml. Particular subjects at risk moreover include subjects prescribed or candidates for a cholesterol or blood pressure reducing treatment or therapy. Specific non-limiting examples of such subjects include candidates from or those being treated with a statin, ACE inhibitor, calcium antagonist, anti-diabetic, or a beta-blocker.

Compositions and methods of the invention may be contacted or provided in vitro, ex vivo or administered in vivo. Compositions and compounds such as a Gal-3BP polypeptide sequence can be administered to provide the intended effect as a single or multiple dosages, for example, in an effective or sufficient amount. Exemplary doses range from about 25-250, 250-500, 500-1000, 1000-2500 or 2500-5000, 5000-25,000, 5000-50,000 pg/kg; from about 50-500, 500-5000, 5000-25,000 or 25,000-50,000 ng/kg; and from about 25-250, 250-500, 500-1000, 1000-2500 or 2500-5000, 5000-25,000, 5000-50,000 mg/kg, on consecutive days, alternating days or intermittently.

Single or multiple doses can be administered on the same or consecutive days, alternating days or intermittently. For example, a compound such as a Gal-3BP polypeptide sequence can be administered one, two, three, four or more times daily, on alternating days, bi-weekly, weekly, monthly, bi-monthly, or annually. Gal-3BP polypeptide sequence can be administered for any appropriate duration, for example, for period of 1 hour, or less, e.g., 30 minutes or less, 15 minutes or less, 5 minutes or less, or 1 minute or less.

Compositions and compounds such as a Gal-3BP polypeptide sequence can be administered to a subject and methods may be practiced substantially contemporaneously with, or within about 1-60 minutes, hours (e.g., within 1, 2, 3, 4 or 5 hours), or days of the onset of an acute or chronic adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

Compositions and compounds such as a Gal-3BP polypeptide sequence can be administered and methods may be practiced via systemic, regional or local administration, by any route. For example, a Gal-3BP polypeptide sequence may be administered systemically, regionally or locally, via injection, via infusion, by catheter, intravenously, intraarterially, orally (e.g., ingestion or inhalation), intramuscularly, intraperitoneally, intradermally, subcutaneously, intracavity, intracranially, transdermally (topical), parenterally, e.g. transmucosally. Compositions and methods of the invention including pharmaceutical formulations can be administered via a (micro)encapsulated delivery system or packaged into an implant for administration (e.g., a coated or impregnated cardiac implant or stent).

Compositions and compounds such as a Gal-3BP polypeptide sequence and methods include pharmaceutical compositions, which refer to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation. Such formulations can be contained in a tablet (coated or uncoated), capsule (hard or soft), microbead, emulsion, powder, granule, crystal, suspension, syrup or elixir.

Pharmaceutical compositions can be formulated to be compatible with a particular route of administration. Compositions for parenteral, intradermal, or subcutaneous administration can include a sterile diluent, such as water, saline solution, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyethylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin can prolonged absorption of injectable compositions.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

Additional pharmaceutical formulations and delivery systems are known in the art and are applicable in the methods of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky, et al., Drug Delivery Systems, R. L. Juliano, ed., Oxford, N.Y. (1980), pp. 253-315).

The compositions used in accordance with the invention, including proteins (e.g., Gal-3BP polypeptide sequence), treatments, therapies, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The invention provides cell-free (e.g., in solution, in solid phase) and cell-based (e.g., in vitro or in vivo) methods of screening, detecting, identifying and quantifying Gal-3BP polypeptide sequence. The methods can be performed in solution, in vitro using a biological material or sample, and in vivo, for example, using a fluid or lavage sample from an animal.

In accordance with the invention, there are provided, methods of diagnosing a subject having or at risk of an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

In one embodiment, a method includes measuring Gal-3BP polypeptide sequence in a sample from a subject, wherein an amount of Gal-3BP polypeptide sequence in the sample below a certain quantity (for example, less than about 10 ug/ml, e.g., 9, 8, 7, 6, 5, 3, 2, or 1 ug/ml) diagnoses the subject as having or at risk of an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

In accordance with the invention, there are also provided, methods of diagnosing a subject protected from an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

In one embodiment, a method includes measuring Gal-3BP polypeptide sequence in a sample from a subject, wherein an amount of Gal-3BP polypeptide sequence in the sample greater than normal (for example, greater than about 10 ug/ml, e.g., 11, 12, 13, 14, or 15 ug/ml, or more) diagnoses the subject as being protected from an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

In various aspects of the methods, measuring Gal-3BP polypeptide sequence includes determining the amount of Gal-3BP polypeptide or nucleic acid encoding Gal-3BP polypeptide (e.g., RNA, cDNA) in the sample. In another aspect, Gal-3BP polypeptide measuring includes contacting the sample with an agent or tag (e.g., a detectable agent or tag, such as an antibody, protein or nucleic acid that binds to Gal-3BP polypeptide or nucleic acid encoding Gal-3BP polypeptide) that binds to Gal-3BP polypeptide or nucleic acid encoding Gal-3BP polypeptide and ascertaining the amount of Gal-3BP polypeptide or nucleic acid encoding Gal-3BP polypeptide, or the amount of agent or tag (e.g., a detectable agent or tag, such as an antibody, protein or nucleic acid that binds to Gal-3BP polypeptide or nucleic acid encoding Gal-3BP polypeptide) bound to the Gal-3BP polypeptide or nucleic acid encoding Gal-3BP polypeptide.

The invention also provides cell-free (e.g., in solution, in solid phase) and cell-based (e.g., in vitro or in vivo) methods of diagnosing and monitoring progression of a subject having or at increased risk of having an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, as well as identifying a subject appropriate for treatment for an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, due to increased probability of developing an adverse cardiovascular event (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation. The methods can be performed in solution, in vitro using a biological material or sample, for example, a sample or biopsy of cells, tissue or organ. The methods can also be performed in vivo, for example, in an animal.

In one embodiment, a method includes contacting a biological material or sample (e.g., from a subject) with an agent that binds to Gal-3BP polypeptide sequence, such as an antibody that binds to Gal-3BP polypeptide or a nucleic acid that hybridizes to a nucleic acid that encodes Gal-3BP polypeptide sequence; and assaying for the presence of Gal-3BP polypeptide. The binding to Gal-3BP polypeptide can be used to ascertain the presence or amount of Gal-3BP polypeptide, which depending on the amount of Gal-3BP polypeptide, is correlated with the presence or increased risk, or absence or decreased risk of developing an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

The presence or amount of Gal-3BP polypeptide less than a certain amount (for example, less than about 10 ug/ml, e.g., 9, 8, 7, 6, 5, 3, 2, or 1 ug/ml) can also identify a subject appropriate for a treatment for an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation, as such subjects will have a greater probability of developing such disorders and diseases.

In one aspect, a biological material or sample is obtained from a mammal (e.g., a human). Methods of diagnosis and measuring Gal-3BP polypeptide can be performed at regular or irregular intervals, for example, daily, bi-weekly, weekly, bi-monthly, monthly, quarterly, semi- or bi-annually, annually, etc., as appropriate, to ascertain changes in risk of developing an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation over time.

Diagnostic methods can be performed on any subject, such as a mammal (e.g., human, primate). Such subjects can be have or be at risk of having an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

The terms “assaying” and “measuring” and grammatical variations thereof are used interchangeably herein and refer to either qualitative or quantitative determinations, or both qualitative and quantitative determinations. When the terms are used in reference to detection, any means of assessing the relative amount is contemplated, including the various methods set forth herein and known in the art. For example, amounts of Gal-3BP polypeptide can be assayed or measured by an ELISA assay, Western blot or immunoprecipitation assay, or by measuring an activity, function or expression of a native Gal-3BP polypeptide sequence. In another example, nucleic acid encoding Gal-3BP, which reflects levels of Gal-3BP polypeptide sequence, can be assayed or measured by hybridization or polymerase chain reaction using probes and primers that bind to nucleic acid encoding Gal-3BP polypeptide sequence.

The term “correlating” and grammatical variations thereof refers to a relationship or link between two or more entities. For example, as disclosed herein relative higher amounts of Gal-3BP polypeptide sequence are associated with protection against adverse cardiovascular events. As also disclosed herein relative low amounts of Gal-3BP polypeptide sequence are associated with the presence of or an increased risk of adverse cardiovascular events and diseases. Thus, because of this relationship between higher amounts of Gal-3BP polypeptide sequence and protection against adverse cardiovascular events, and lower amounts of Gal-3BP polypeptide sequence and increased risk of adverse cardiovascular events and cardiovascular diseases, Gal-3BP polypeptide levels and adverse cardiovascular events and cardiovascular diseases correlate with each other. Thus, correlating the quantity of Gal-3BP polypeptide sequence can indicate susceptibility or increased risk of, as well as decreased risk, in a subject, for example, of developing an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

The invention provides kits including compositions of the invention (e.g., Gal-3BP polypeptide sequence, etc.), combination compositions and pharmaceutical formulations thereof, packaged into suitable packaging material. Kits can be used in various methods. For example, a kit can determine an amount of Gal-3BP polypeptide sequence, since Gal-3BP polypeptide sequence reflects protection from an adverse cardiovascular event or cardiovascular disease, etc., or an increased risk of an adverse cardiovascular event, etc. The amount of Gal-3BP polypeptide sequence in blood or other body fluids would indicate relative protection or the risk of an adverse cardiovascular event, cardiovascular disease, etc. Alternatively, kits may detect splice variants, proteolytic products, truncated products or post-translational modifications of Gal3-BP.

A kit typically includes a label or packaging insert including a description of the components or instructions for use in vitro, in vivo, or ex vivo, of the components therein. A kit can contain a collection of such components, e.g., Gal-3BP polypeptide sequence, antibody that binds to Gal-3BP polypeptide sequence, alone, or in combination with another therapeutically useful composition (e.g., a blood pressure or cholesterol lowering drug).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Exemplary instructions include, instructions for treating an adverse cardiovascular event or cardiovascular disease (e.g., coronary artery disease, peripheral artery disease, cerebrovascular disease, renal artery disease, stroke, myocardial infarction (heart attack), ischemic heart failure, transient ischemic attack or brain trauma), artherosclerotic plaque formation, foam cells or foam cell formation.

Kits of the invention therefore can additionally include labels or instructions for practicing any of the methods of the invention described herein including treatment, or diagnostic and detection methods.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Invention kits can additionally include other components. Each component of the kit can be enclosed within an individual container and all of the various containers can be within a single package. Invention kits can be designed for cold storage. Invention kits can further be designed to contain Gal-3BP polypeptide sequences or antibodies, or that contain nucleic acid that binds (hybridizes) to nucleic acid encoding Gal-3BP polypeptide.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “a Gal-3BP polypeptide sequence” includes a plurality of such Gal-3BP polypeptide sequences or subsequences thereof, and reference to “an Gal-3BP polypeptide activity or function” can include reference to one or more Gal-3BP polypeptide activities or functions, and so forth.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 2-10 includes 2, 3, 4, 5, 6, 7, 8, 9, 10, as well as 1.1, 1.2, 1.3, 1.4, 1.5, etc., 2.1, 2.2, 2.3, 2.4, 2.5, etc., and any numerical range within such a ranges, such as 2-3, 2-4, 2-6, 3-6, 3-7, 4-8, 5-9, 5-10, etc. In a further example, reference to a range of 2-10, 10-20, 20-30, 30-40, 40-50, 50-60, 60-75, 75-100 includes any numerical value or range within or encompassing such values.

As also used herein a series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 2-10, 10-20, 20-30, 30-40, 40-50, 50-60, 60-75, 75-100, includes ranges such as 2-20, 2-30, 5-20, 5-30, 5-40, 5-50, 5-60, 10-30, 10-40, 10-50, and 20-40, 20-30, 20-40, 20-50, 30-50, 30-60, 30-100, and 40-60, 40-70, 40-100, 50-75, 50-100, 60-100, and so forth.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of various materials and methods.

### Monocyte-Derived Macrophages

Monocytes were isolated from peripheral blood of healthy donors by density gradient centrifugation (Histopaque, Sigma Aldrich, St. Louis, Mo.) and subsequent negative isolation using magnetic beads (Miltenyi). Monocyte purity as assessed by flow cytometry for CD14 (clone HCD14, BioLegend, San Diego, Calif.) was routinely >97%. Monocytes were cultured for six days with M-CSF (100 ng/ml, Peprotech, Rocky Hill, N.J.) in macrophage serum-free medium (Gibco, Carlsbad, Calif.) supplemented with nutridoma SP (Roche, Nutley, N.J.), and penicillin/streptomycin (Sigma Aldrich). After this period, cells displayed the expected macrophage-like shape and were all positive for macrophage markers like CD68 (clone Y1/82A, BD Biosciences) or CD 11b (clone CR3, BD Biosciences). Blood draws were part of the Normal Blood Donor Program.

### Microparticle Mass Spectrometry

Blood samples (20 ml in ACD) were collected from eleven patients with angiographically verified coronary artery disease. After centrifugation for 10 minutes in a tabletop centrifuge to remove blood cells, plasma was centrifuged twice more at 710 g for 15 minutes at room temperature, discarding the pellet each time. The sample was processed immediately into microparticles by gel filtration and ultracentrifugation as described in reference (Smalley et al., Thromb. Hemost. 97:67-80 (2007)), passed over a C18 HPLC column (gradient) directly into a Thermo Finnegan LTQ-FT tandem mass spectrometer. All MS2 data were searched against a human protein database downloaded from the European Bioinformatics Institute (www.ebi.ac.uk) using SEQUEST (Thermo, Waltham, Mass.). A static modification of 57.02150 Da for cysteine residues, and variable modifications of 15.9949 and 14.01550 for methionine residues and cysteine residues, respectively, were allowed. The parent mass tolerance was set to 10 ppm and the mass tolerance of daughter ions was set at 0.5 Da. Peptide identifications were made based on fully tryptic peptides, using a first-pass filtering criteria requiring cross correlation values of 1.8, 2.3, and 2.5 for charged states of +1, +2, and +3, respectively. Peptide and protein identifications from MS2 spectra were determined using SEQUEST (Thermo).

### Gene Chip Studies and Quantitative PCR

Total RNA was isolated from cultured macrophages and foam cells using the RNEasy Mini Kit with DNase treatment (all Qiagen, Valencia, Calif.). Gene chip studies were performed at the Gene Chip/Microarray Bioinformatics Core at the University of Virginia. Labeling of samples, hybridization, and scanning were performed according to standard Affymetrix protocols.

Microarray gene expression intensities were normalized to ensure equal inter-quartile ranges (IQR), log 2-transformed, and analyzed by open source statistical software package R (www.rproject.org). The heterogeneous error model (HEM) (Cho and Lee, Bioinformatics 20:2016 (2004)) was applied for differential expression discovery under multiple conditions and the local pooled error (LPE) test (Jain et al., Bioinformatics 19:1951 (2003)) for differential expression discovery under two conditions. Signal intensity values were obtained from the Affymetrix MicroArray Suite software (MAS 5.0). Of 22,283 probe sets on the HG-U133A chip, 78 internal control probes were removed and 22,215 probe sets representing 12,978 gene products were analyzed. Gene expression data have been deposited at the NCBI Gene Expression Omnibus database (series GSE7138) (Cho et al., Physiol. Genomics 29:149 (2007)). Non-expressed (within 2 SD from zero in all conditions) and housekeeping genes (not regulated) were eliminated from further analysis.

For quantitative PCR, reverse transcription was performed with the Omniscript RT Kit (Qiagen). Gene expression was measured using Sybr Green (Roche). Primers were purchased from Invitrogen. Primer sequences were obtained from Primer bank (Wang and Seed, Nucleic Acids Res. 31:e154 (2003)) and are shown in Table 1. All samples were run in duplicates. Gene expression was calculated using GAPDH as housekeeping gene.

TABLE 1
Primer sequences used for real-time PCR

| Gene | | | Product |
|---|---|---|---|
| symbol | Forward sequence | Reverse sequence | length |
| GAPDH | GGCTCATGACCACAGTCCAT | GCCTGCTTCACCACCTTCT | 277 by |
| CD36 | GCCAAGGAAAATGTAACCCAGG | GCCTCTGTTCCAACTGATAGTGA | 101 by |
| MSR1 | GCAGTGGGATCACTTTCACAA | CTTGAAGGGAAGGGCTGTTTT | 125 by |
| ABCA1 | AACTCTACATCTCCCTTCCCG | CTCCTGTCGCATGTCACTCC | 123 by |
| ABCG1 | ACGCAGGGGTGGACAAAAC | GAGACACACACCGACTTGGG | 92 by |

### Flow Cytometry

For flow cytometry, human cells were treated with Fc block (Miltenyi, Auburn, Calif.) and stained with antibodies against CD36 (clone CB38, BD Biosciences, San Jose, Calif.) and SR-A (clone 351615, R&D Systems, Minneapolis, Minn.). For SR-A staining, a FITC-labeled secondary antibody was used. For mouse studies, the following antibodies were used: anti-Gr1-APC (clone RB6-8C5), anti-CD45-PerCP (clone 30-F11, Becton Dickinson), anti-CD11b-Pacific Blue (clone M1/70, eBioscience, San Diego, Calif.), anti-MHC-II Alexa 700 (M5/114.15.2) (both eBioscience, San Diego, Calif.) and CD36-Alexa 488 (HM36, Biolegend). Aqua LIVE/DEAD® Fixable Dead Cell Stain Kit was used in all experiments (Invitrogen, Carlsbad, Calif.). Appropriate isotype controls and fluorescence-minus-one (FMO) controls were used in all experiments. Fluorescence was measured either on a FACSCalibur or a Becton Dickinson LSRII flow cytometer (Becton Dickinson). Fluorescence was assessed as background-corrected mean fluorescence (MFI).

### Galectin-3 Binding Protein ELISA

Gal-3BP plasma levels were analyzed using a commercially available ELISA, which applies two monoclonal antibodies against Gal-3BP (Bender MedSystems, Burlingame, Calif.). All measurements were done in duplicate according to the manufacturer's instructions, blinded and in random order to reduce bias due to inter-assay variation. Intra- and inter-assay variances were 5.0% and 9.6%, respectively.

### Cholesterol Loading Studies

Human blood monocyte-derived macrophages were treated with Gal-3BP (recombinantly expressed in a mouse myeloma cell assuring appropriate glycosylation (Laferte et al., J. Cell. Biochem. 77:559 (2000)) of the recombinant protein, R&D Systems) at different concentrations of vehicle for 24 hours and subsequently exposed to acetylated (acLDL) or oxidized LDL (oxLDL) labeled with 1,1-dioctadecyl-3,3,3,3-tetramethylindocarbocyanine perchlorate (DiI) at a concentration of 10 μg/ml (both Biomedical Technologies, Stoughton, Mass.). After four hours, cells were harvested and DiI fluorescence was assessed using a FACSCalibur flow cytometer (BD Biosciences). Fluorescence was quantified as background-corrected mean fluorescence (MFI).

### Plasma Samples

Arterial blood samples were obtained from 94 consecutive patients undergoing coronary angiography. Plasma samples were obtained from the sidearm or the femoral arterial sheath prior to heparin administration and performance of the coronary angiogram. The blood was collected in a purple top tube, placed on ice, and immediately centrifuged. The plasma was collected and stored at −80° C. until analyzed. Seventy-seven patients were included in the present study based on the angiographic diagnosis of obstructive coronary artery disease (CAD) defined as ≧70% luminal diameter narrowing of a major epicardial artery or their major branches. Complete clinical, angiographic, laboratory, and clinical follow-up data was available for all patients.

### Immunohistochemistry

Human coronary arteries were obtained from the University of Virginia Department of Pathology/Tissue bank (Charlottesville, Va.). Coronary arteries were embedded in paraffin and 5 μm sections were prepared. After heat-induced antigen retrieval using antigen unmasking solution (Vector Laboratories), sections were either incubated with antibodies against CD68 (clone KP-1, Santa Cruz Biotechnology, Santa Cruz, Calif.) or Gal-3BP (clone SP-2, Bender MedSystems). Antibodies were detected using the ABC method (Vector Laboratories, Burlingame, Calif.). Antibodies were visualized using DAB (DAKO corporation, Carpinteria, Calif.). Cells were counterstained with hematoxylin (Sigma Aldrich). Sections for immunofluorescence were prepared in the same way. For staining, antibodies against smooth muscle q actin (Cy3-conjugated, clone 1A4, Sigma Aldrich), CD68 (rabbit polyclonal, goat anti-rabbit Texas red as secondary, both Santa Cruz), and Gal-3BP (FITC-conjugated, clone SP-2, Bender MedSystems) and appropriate isotype controls were used. DAPI (Millipore, Billerica, Mass.) was used as nuclear stain.

All sections were analyzed using a Nikon 80i microscope and Image Pro Plus 3.0 software (Media Cybernetics, Bethesda, Md.).

### Production of Recombinant Gal-3BP

For human Gal3-BP, the coding sequence was amplified by PCR using pCMV6-XL4-LGALS3BP (OriGene, Rockville, Md.) as template. The sense primer was 5′-TAGACATATGACCCCTCCGAGGCTCTT-3′ containing a NdeI restriction site (underlined), the anti-sense primer was 5′-ATCAGGATCCTTACTTGTCATCGTCGTCCTTGTAGTCGTCCACACCTGAGGAGTT-3′ containing one copy of FLAG encoding sequence fused in-frame to the last amino acid of human LGALS3BP, followed by a stop codon and a BamHI restriction site (underlined). The PCR products were purified and cloned into pET19b (Novagen, Nottingham, UK).

Rat LGALS3BP was cloned into pET19b using a similar strategy, with the sense primer 5′-GACACATATGGCTCTTCTGTGGCTCCTCT-3′ containing a NdeI restriction site (underlined) and the anti-sense primer 5′-TCGGATCCTTAGTTGGTGAGGTAGAAGGGGC-3′ containing a BamHI restriction site (underlined). The final PCR product was designed to delete the last 5 amino acids to simulate the C-terminus of the mouse Lgals3bp. The sequence and the open reading frame of each plasmid were confirmed by sequencing (Retrogen, San Diego, Calif.). The recombinant proteins were induced and purified through a HIS-Select HF Nickel Affinity Gel as instructed by the manufacturer (Sigma Aldrich).

### Animals

Wild-type (wt) C57Bl/6 mice were from Jackson Labs (Bar Harbor, Me.) and CYCAP KO (Lgals3bp^{-/-}) mice were provided by Iry Weissman (Stanford). Mice were kept in specific-pathogen-free conditions in a barrier facility. For induction of peritonitis, 1 ml BBL fluid thioglycollate medium (Becton Dickinson) was injected intraperitoneally. 2.3 μg purified recombinant rat Gal-3BP or control buffer were injected at time points 0 and 48 hours. DiI-labeled oxidized or acetylated LDL (20 μl acLDL; 200 μg/ml) was injected after 48 hours. After 72 hours, cells were recovered by washing twice with 5 ml PBS. Total cell counts were obtained by an automatic analyzer (Hemavet 950FS, DREW Scientific, Oxford, Conn.). Mouse macrophages and DC were obtained from femur and tibia of CYCAP KO (Lgals3bp^{-/-}) or WT mice and incubated for 6 days with GM-CSF (30% from R1 medium in complete RPMI) using standard methods (Mosser & Edwards, Nat. Rev. Immunol. 8, 958-969 (2008)). These cells were incubated with interferon-γ (250n/ml) for 1 day to produce M1 macrophages or with IL-4 (20 μg/ml) to produce M2 macrophages or with PGE-2 (1 μM) to produce Mreg macrophages and then challenged with and LPS (10 ng/ml) for another day.

### Statistical Analyses

Statistical analyses were performed using Prism (GraphPad, La Jolla, Calif.) and MedCalc (MedCalc, Mariakerke, Belgium). P values were two-tailed and P<0.05, 0.01, or 0.001 are reported. Confidence intervals were calculated at the 95% level. All data are presented as means±standard error (SEM). Paired t tests were used to compare gene or protein expression in cell culture studies. One way ANOVA with post hoc Dunnett's test was used when several groups were compared. For analysis of patient data, differences in mean of continuous variables were compared between groups using t test. Non-parametric Whitney-Mann testing was applied wherever D'Agostino-Pearson normality testing revealed a non-Gaussian distribution. For categorical variables, Fisher's exact test was applied. Receiver-operator characteristics (ROC) analyses were performed to identify optimal cut-off values. Kaplan-Meier survival estimates were calculated for adverse cardiovascular events within 52 weeks using the best cut-off value identified by ROC analysis. Statistical analysis of event-free survival was done by Log-rank (Mantel-Cox) Test. Logistic regression was done including those risk factors used in secondary prevention of cardiovascular disease patients (Smith et al., Circulation 113: 2363 (2006)) and all parameters that were differentially distributed between patients with and without major adverse cardiac events.

### Example 2

This example describes screening studies of a potential role of Gal-3BP in atherosclerosis.

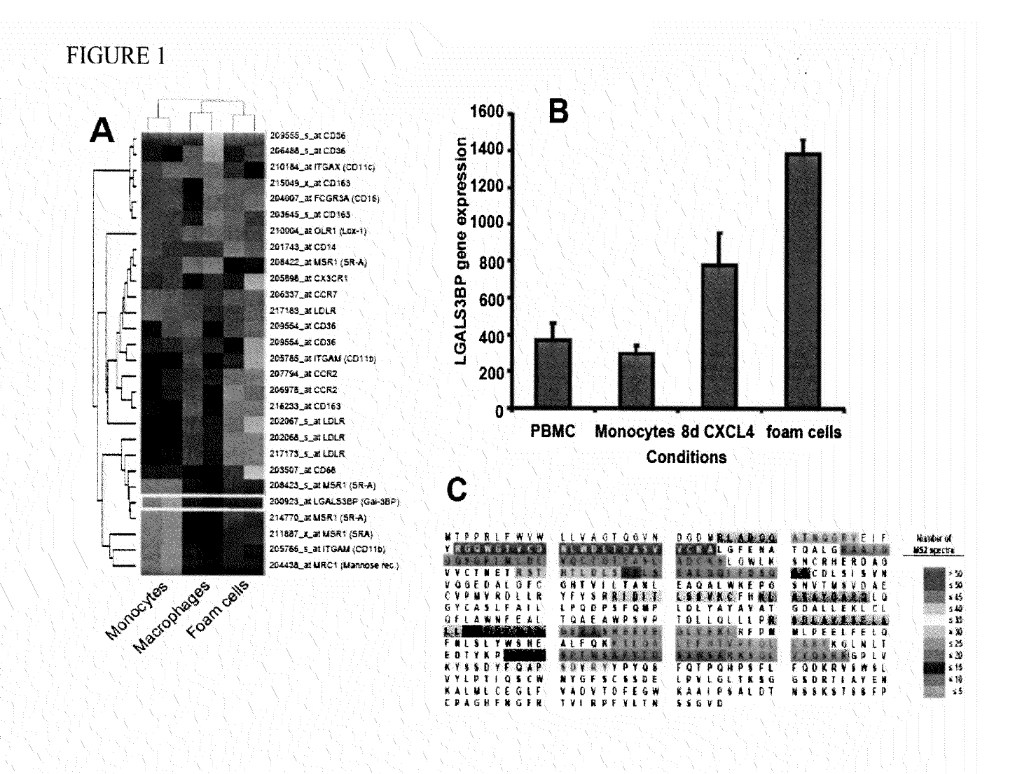

In an Affymetrix gene expression screen (Cho et al., Physiol. Genomics 29: 149 (2007)), mRNA expression of LGALS3BP—the gene coding for Gal-3BP—was significantly upregulated during macrophage foam cell formation (FIGS. 1A,B). This pattern was shared by CD36, MSR1 (scavenger receptor-A [SR-A]) and ITGAM (CD11b), but not by LDLR (LDL receptor), other scavenger receptors or chemokine receptors (FIG. 1A and Table 2).

TABLE 2
Gene expression of monocyte/macrophage surface receptors during foam
cell formation as determined by Affymetrix gene chip analysis.

| | Monocytes | Monocytes | Macrophages | Macrophages | Foam cells | Foam cells |
|---|---|---|---|---|---|---|
| Annotation | chip A | chip B | chip A | chip B | Chip A | Chip B |
| 200923_at LGALS3BP | 328.42 | 262.75 | 1519.15 | 920.01 | 1442.22 | 1335.37 |
| 201743_at CD14 | 12808.82 | 14806.50 | 295.40 | 180.33 | 413.59 | 502.05 |
| 202067_s_at LDLR | 49.20 | 58.99 | 92.96 | 105.55 | 2.35 | 8.01 |
| 202068_s_at LDLR | 572.16 | 625.07 | 578.64 | 649.42 | 90.23 | 97.25 |
| 203104_at CSF1R | 2666.98 | 2413.27 | 484.02 | 332.66 | 533.36 | 297.02 |
| 203507_at CD68 | 343.41 | 284.64 | 191.11 | 191.61 | 157.73 | 330.60 |
| 203645_s_at CD163 | 350.34 | 420.34 | 104.70 | 48.11 | 77.75 | 47.20 |
| 204007_at FCGR3A | 674.52 | 975.12 | 127.55 | 74.47 | 151.87 | 158.72 |
| 204438_at MRC1 | 77.48 | 67.78 | 3912.24 | 2549.76 | 4884.98 | 4937.34 |
| 205785_at ITGAM | 120.74 | 137.93 | 60.35 | 43.90 | 48.21 | 83.86 |
| 205786_s_at ITGAM | 1289.80 | 1315.83 | 2746.96 | 1995.74 | 2882.74 | 3015.72 |
| 205898_at CX3CR1 | 284.03 | 311.84 | 554.23 | 607.77 | 269.40 | 241.37 |
| 206337_at CCR7 | 605.39 | 785.04 | 921.30 | 1560.20 | 605.56 | 688.43 |
| 206488_s_at CD36 | 308.07 | 315.47 | 243.67 | 95.60 | 336.48 | 218.87 |
| 206978_at CCR2 | 202.31 | 166.80 | 116.33 | 122.27 | 111.24 | 74.53 |
| 207794_at CCR2 | 99.53 | 142.11 | 43.94 | 47.70 | 43.51 | 43.90 |
| 208422_at MSR1 | 14.56 | 57.04 | 21.29 | 31.49 | 5.12 | 13.39 |
| 208423_s_at MSR1 | 96.84 | 97.07 | 446.21 | 264.44 | 365.81 | 359.47 |
| 209554_at CD36 | 54.71 | 29.29 | 14.75 | 10.57 | 10.45 | 24.70 |
| 209555_s_at CD36 | 235.96 | 260.66 | 241.28 | 175.00 | 345.65 | 320.29 |
| 210004_at OLR1 | 8529.13 | 8863.15 | 79.56 | 35.59 | 93.32 | 61.25 |
| 210184_at ITGAX | 2355.38 | 640.27 | 821.07 | 878.78 | 1017.01 | 1543.59 |
| 211887_x_at MSR1 | 57.26 | 48.11 | 467.40 | 358.30 | 642.46 | 487.13 |
| 214770_at MSR1 | 70.97 | 65.69 | 503.55 | 396.97 | 618.78 | 704.13 |
| 215049_x_at CD163 | 301.28 | 359.26 | 107.40 | 99.09 | 88.41 | 78.04 |
| 216233_at CD163 | 6.36 | 8.37 | 5.09 | 5.03 | 4.27 | 3.95 |
| 217173_s_at LDLR | 124.56 | 156.34 | 117.89 | 120.22 | 55.88 | 55.65 |
| 217183_at LDLR | 14.14 | 12.27 | 7.58 | 8.10 | 9.17 | 9.88 |

Using liquid chromatography electrospray tandem mass spectrometry (LC-MS/MS), Gal-3BP was enriched in plasma microparticles in eleven patients with angiographically confirmed obstructive coronary artery disease (CAD). Microparticles are known to be shed from atherosclerotic lesions (Chironi et al., Arterioscler Thromb Vasc Biol 26: 2775 (2006)). Most of the microparticles in healthy subjects are thought to be derived from activated platelets, but some are from leukocytes and endothelial cells (Smalley, D. M. & Ley, K., Clin. Lab. 54: 67 (2008)), cells involved in the development of atherosclerotic lesions. Sixteen unique Gal-3BP-derived peptides were identified in 384 tandem mass spectra, proving excellent coverage and high confidence detection of Gal-3BP (FIG. 1C).

### Example 3

This example shows the effect of recombinant human Gal-3BP on cholesterol uptake.

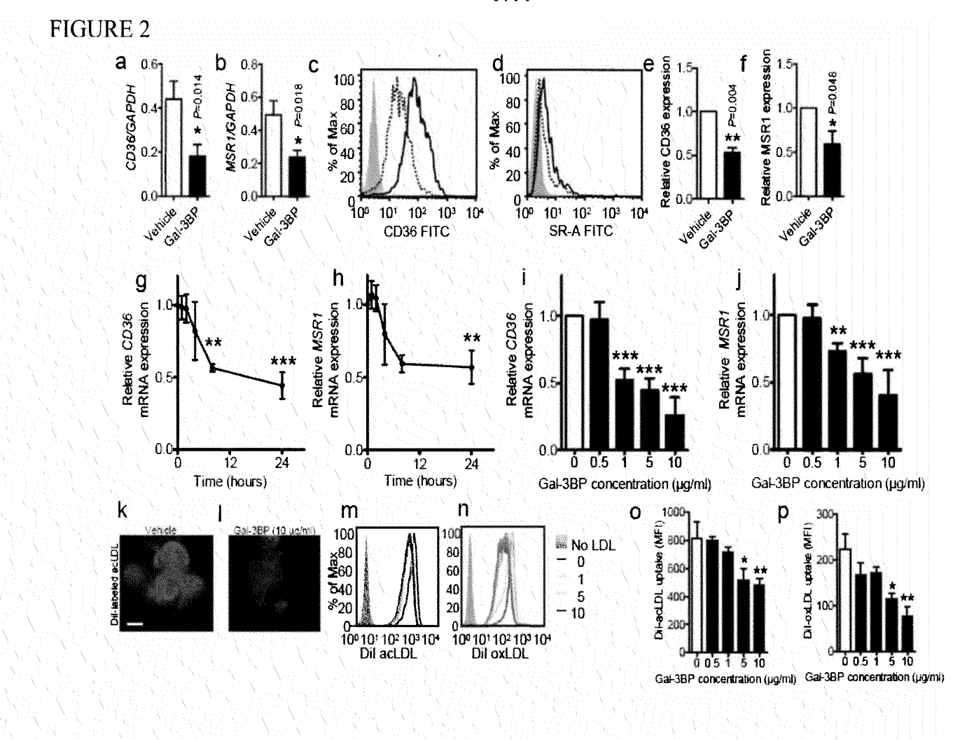

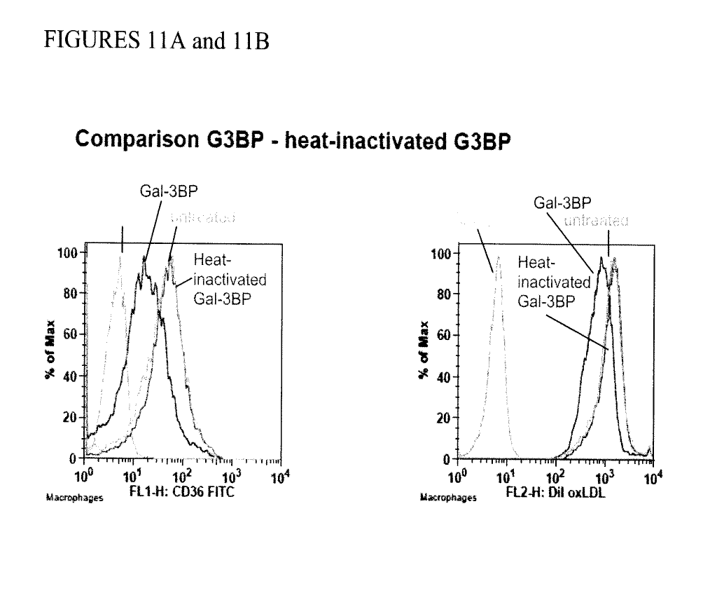

To assess whether treatment of human blood monocyte-derived macrophages with recombinant human Gal-3BP affects expression of receptors relevant for cholesterol uptake, CD36 and MSR1 (scavenger receptor-A [SR-A]) mRNA (FIGS. 2A,B) and CD36 and SR-A cell surface protein (FIGS. 2C-F) were analyzed. Both mRNA and cell surface protein levels were significantly downregulated after 24 hours treatment with 5 μg/ml human recombinant Gal-3BP as compared to vehicle-treated controls. The effect of Gal-3BP on CD36 and SR-A expression was time-dependent (FIGS. 2G,H) and increased with dose (FIGS. 2I,J), reaching a maximum at 10 μg/ml. CD36 and SR-A combined account for the vast majority of modified LDL uptake (Kunjathoor et al., J. Biol. Chem. 277:49982 (2002)). Absence of SR-A (Babaev et al., Arterioscler. Thromb. Vasc. Biol. 20:2593 (2000)) or CD36 (Febbraio et al., J. Clin. Invest. 105:1049 (2000)) reduces atherogenesis in Apoe^{-/-} mice. A mouse model of atherosclerosis lacking both the MsrI (SR-A) and the Cd36 genes showed no additive effect (Kuchibhotla et al., Cardiovasc. Res. 78: 185 (2008)), but lesion complexity and the number of apoptotic cells within the lesions rather were significantly reduced (Manning-Tobin et al., Arterioscler. Thromb. Vasc. Biol. 29: 19 (2009)). Suppression of foam cell formation by Gal-3BP could have beneficial effects in vivo. When human primary macrophages were treated with Gal-3BP for 24 hours and then exposed to DiI-labeled acetylated LDL (acLDL; 10 μg/ml, FIGS. 2K,L), uptake of acetylated LDL was reduced by pre-incubation of macrophages with Gal-3BP (FIGS. 2M,N) in a dose-dependent manner (FIGS. 2O,P). This effect was absent when Gal-3BP was denaturized by incubation at 80° C. for 20 minutes (FIGS. 11A,B). The difference in oxLDL content between Gal-3BP-treated and vehicle-treated cells remained similar over time, suggesting that Gal-3BP persistently inhibits rather than delays oxLDL uptake.

### Example 4

This example shows the effects of Gal-3BP deficiency on macrophage differentiation and foam cell formation.

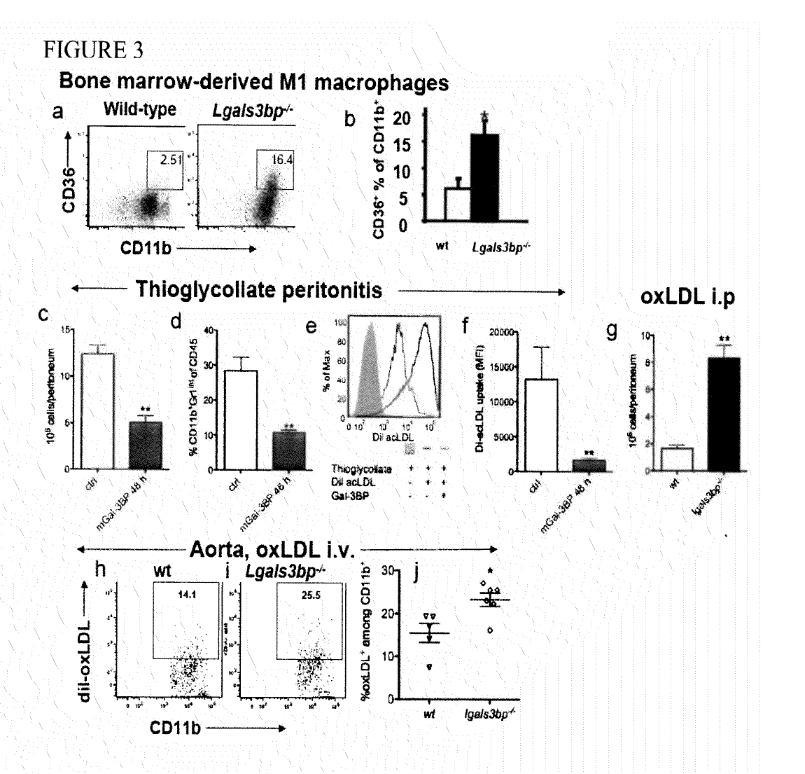

To assess the effects of Gal-3BP deficiency on macrophage differentiation and foam cell formation in vivo, we employed Lgals3bp^{-/-} mice, which do not express CyCAP, the homologue of human Gal-3BP (Trahey, M. & Weissman, I. L., Proc. Natl. Acad. Sci. USA 96, 3006 (1999)). Bone-marrow derived macrophages from these mice differentiated under M1 polarizing conditions (LPS, IFN-γ) (Martinez et al., J Immunol 177: 7303 (2006)) expressed similar levels of CD11b, but significantly higher levels of CD36 (FIG. 3A), and the percentage of CD36⁺ macrophages was significantly increased (FIG. 3B), suggesting that autocrine secretion of murine Gal-3BP suppresses CD36 expression in human macrophages. This effect was not seen in macrophages differentiated with M-CSF (M0), M-CSF- and IL-4 (M2), or M-CSF and PGE2 (Mreg) (Martinez et al., J Immunol 177: 7303 (2006); and Mosser, D. M. & Zhang, X., Curr Protoc Immunol Chapter 14: Unit 14 12 (2008)).

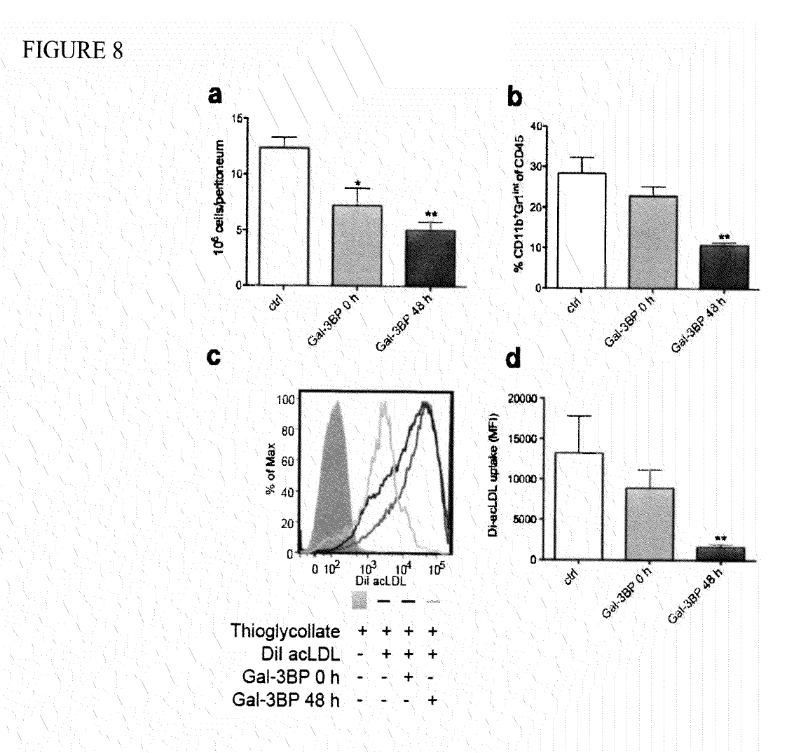

Three mouse models were used to study in vivo effects of recombinant Gal-3BP on macrophage accumulation and foam cell formation in vivo. mGal-3BP (2.3 μg) or control buffer were injected i.p. together with DiI-labeled acLDL into wild type mice when thioglycollate was administered at 0 hours or at 48 hours after thioglycollate, and peritoneal macrophages were harvested at 72 hours (FIGS. 3C-3F and FIG. 8). Injection of Gal-3BP significantly reduced the absolute number of peritoneal cells (FIG. 3C) as well as the percentage of CD11b⁺Gr1^{int} positive macrophages (FIG. 3D). Gal-3BP significantly inhibited foam cell formation as measured by reduced uptake of DiI-acLDL (FIG. 3E,F). When oxLDL was injected intraperitoneally without thioglycollate, Lgals3bp^{-/-} mice, but not wild type mice recruited macrophages (FIG. 3G). These results demonstrate that endogenous baseline Gal-3BP inhibits macrophage accumulation and foam cell formation in vivo.

Since the focus of the clinical study was on atherosclerosis, the relevant organ system in which foam cell formation leads to disease is the arterial system. When Lgals3bp^{-/-} and wild type mice were injected with DiI-labeled oxLDL i.v., significantly more oxLDL was observed in CD11b⁺ aortic macrophages as measured by flow cytometry (FIGS. 3H,I) in Lgals3bp^{-/-} than in wild type mice (FIG. 3J).

### Example 5

This example shows Gal-3BP mediated protection from adverse cardiovascular events in patients with angiographically confirmed obstructive CAD.

To study whether Gal-3BP-induced inhibition of foam cell formation was clinically relevant, plasma levels in 77 patients with angiographically confirmed obstructive CAD (Table 3) were measured and compared with plasma levels in age-matched controls with angiographically confirmed absence of significant CAD (n=17, age 41-83 years) as well as young healthy individuals (n=23, age 24-32 years).

TABLE 3
Demographic and clinical characteristics of patients with or without
MACE as defined by death from cardiovascular disease, myocardial
infarction, cerebrovascular incident, or need for interventional or
surgical revascularization.

| | Value (n = 77) |
|---|---|
| Parameter | |
| Gender (% male) | 65 |
| Age (years) | 59.2 ± 1.2 |
| Heart rate (1/min) | 67 ± 1.3 |
| Systolic blood pressure (mmHg) | 138 ± 3 |
| Diastolic blood pressure (mmHg) | 70 ± 1 |
| Blood work | |
| Triglycerides (mg/dL) | 184 ± 14 |
| Total cholesterol (mg/dL) | 181 ± 6 |
| LDL (mg/dL) | 107 ± 5 |
| HDL (mg/dL) | 39 ± 1 |
| White blood count (1/nL) | 7.5 ± 0.3 |
| Platelet count (1/nL) | 253.4 ± 8.1 |
| Plasma creatinine (mg/dL) | 1.1 ± 0.1 |
| CRP (mg/L) | 11.8 ± 1.9 |
| Current medical therapy | |
| ASA (%) | 99 |
| Clopidogrel (%) | 9 |
| ACE inhibitor (%) | 66 |
| Calcium antagonist (%) | 25 |
| β blocker (%) | 84 |
| Statin (%) | 62 |
| Oral anti-diabetic drugs (%) | 17 |
| Insulin (%) | 22 |
| Current treatment | |
| PCI (%) | 39 |
| Stent (%) | 29 |
| Prior medical history | |
| History of diabetes mellitus (%) | 42 |
| History of hyperlipidemia (%) | 77 |
| History of smoking (%) | 52 |
| Family history of cardiovascular disease (%) | 46 |
| History of peripheral vascular disease (%) | 26 |
| Previous myocardial infarction (%) | 40 |
| Previous CABG (%) | 14 |
| Previous PCI (%) | 26 |

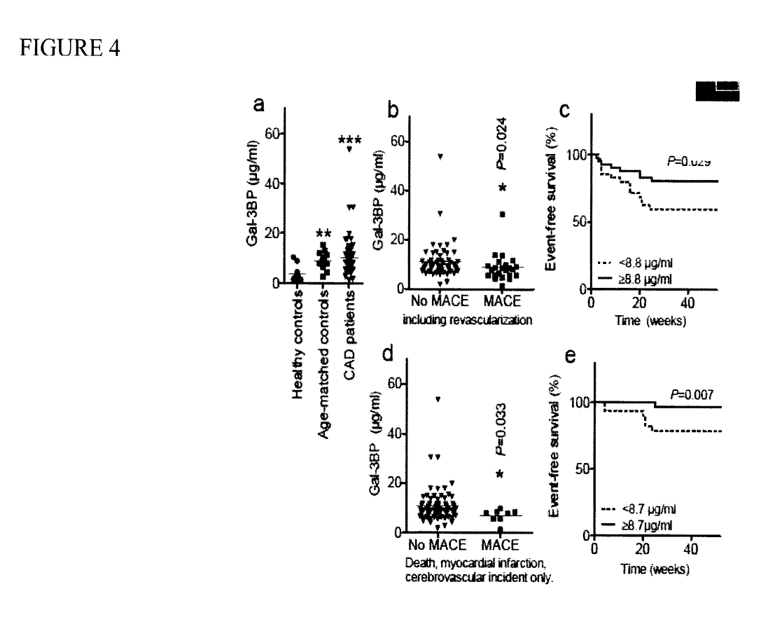

Patients with obstructive cardiovascular disease displayed high levels of Gal-3BP (10.6±0.8 μg/ml), similar to age-matched controls (9.1±0.7 μg/ml, P<0.01 by non-parametric Mann-Whitney test) and much higher than young adults (3.9±0.8 μg/ml, P<0.0001 by non-parametric Mann-Whitney test, FIG. 4A). To determine whether Gal-3BP was associated with clinical outcomes in CAD, Gal-3BP plasma levels and major adverse cardiac events (MACE) were studied in the 77 CAD patients within one year after coronary angiography. MACE were defined as death from cardiovascular cause, myocardial infarction, cerebrovascular incident, or need for surgical or percutaneous coronary artery revascularization (Table 4).

TABLE 4
Demographic and clinical characteristics of patients with or without
MACE as defined by death from cardiovascular disease, myocardial
infarction, or cerebrovascular incident, and need for interventional or
surgical revascularization.

| | MACE | No MACE | |
|---|---|---|---|
| | (n = 23) | (n = 54) | P value |
| Parameter | | | |
| Gender (% male) | 22 | 41 | 0.126 |
| Age (years) | 56.3 ± 1.9 | 60.5 ± 1.5 | 0.096 |
| Heart rate (1/min) | 65 ± 2 | 68 ± 2 | 0.226 |
| Systolic blood pressure (mmHg) | 132 ± 5 | 140 ± 4 | 0.149 |
| Diastolic blood pressure (mmHg) | 71 ± 2 | 70 ± 2 | 0.730 |
| Blood work | | | |
| Triglycerides (mg/dL) | 212 ± 30 | 172 ± 16 | 0.381 |
| Total cholesterol (mg/dL) | 189 ± 10 | 178 ± 7 | 0.302 |
| LDL (mg/dL) | 109 ± 8 | 107 ± 6 | 0.527 |
| HDL (mg/dL) | 38.7 ± 2.5 | 38.9 ± 1.7 | 0.667 |
| White blood count (1/nL) | 7.2 ± 0.4 | 7.6 ± 0.3 | 0.670 |
| Platelet count (1/nL) | 244.2 ± 13.5 | 257.4 ± 10.1 | 0.520 |
| Plasma creatinine (mg/dL) | 1.1 ± 0.1 | 1.1 ± 0.1 | 0.637 |
| CRP (mg/L) | 8.6 ± 3.3 | 13.2 ± 2.3 | 0.213 |
| Current medical therapy | | | |
| ASA (%) | 100 | 98 | 1.000 |
| Clopidogrel (%) | 9 | 9 | 1.000 |
| ACE inhibitor (%) | 48 | 74 | 0.036 |
| Calcium antagonist (%) | 22 | 26 | 0.780 |
| β blocker (%) | 83 | 85 | 0.744 |
| Statin (%) | 39 | 72 | 0.010 |
| Oral anti-diabetic drugs (%) | 4 | 22 | 0.093 |
| Insulin (%) | 13 | 19 | 0.248 |
| Current treatment | | | |
| PCI (%) | 26 | 44 | 0.201 |
| Stent (%) | 13 | 35 | 0.058 |
| Prior medical history | | | |
| History of diabetes mellitus (%) | 22 | 50 | 0.025 |
| History of hyperlipidemia (%) | 70 | 80 | 0.384 |
| History of smoking (%) | 61 | 48 | 0.331 |
| Family history of cardiovascular | 48 | 44 | 0.807 |
| disease (%) | | | |
| History of peripheral vascular | 26 | 26 | 1.000 |
| disease (%) | | | |
| Previous myocardial infarction (%) | 35 | 43 | 0.616 |
| Previous CABG (%) | 13 | 15 | 1.000 |
| Previous PCI (%) | 30 | 24 | 0.580 |

Mean Gal-3BP plasma levels were significantly lower in patients with MACE (8.8±1.7 μg/ml versus 11.3±1.0 μg/ml in no MACE patients; P=0.024 by non-parametric Mann-Whitney test; FIG. 4B). Receiver operator curve (ROC) analysis yielded an area under the curve (AUC) of 0.66 (95% CI 0.53-0.80; P=0.024), and suggested an optimal cut-off of 8.8 μg/ml with a sensitivity of 63.0% and a specificity of 65.2%. Kaplan-Meier analysis of CAD patients using a cut-off of 8.8 μg/ml demonstrated a good discriminative power of Gal-3BP plasma levels with a negative predictive value of 77.1% (P=0.029, hazard ratio 2.5 [95% CI 1.1-5.9]; FIG. 4C). Patients reaching a more stringent endpoint defined as death from cardiovascular cause, myocardial infarction, or cerebrovascular incident (Table 5) had a mean Gal-3BP plasma level of 7.0±0.9 μg/ml, compared to 11.0±0.9 μg/ml in patients who remained event-free during follow-up (P=0.033 by non-parametric Mann-Whitney test; FIG. 4D).

TABLE 5
Demographic and clinical characteristics of patients with or without
MACE as defined by death from cardiovascular disease, myocardial
infarction or cerebrovascular incident.

| | MACE | No MACE | |
|---|---|---|---|
| | (n = 8) | (n = 61) | P value |
| Parameter | | | |
| Gender (% male) | 100 | 60.9 | 0.045 |
| Age (years) | 54.8 ± 2.8 | 59.7 ± 1.3 | 0.226 |
| Heart rate (1/min) | 67 ± 4 | 67 ± 1 | 0.933 |
| Systolic blood pressure (mmHg) | 121 ± 6 | 140 ± 3 | 0.036 |
| Diastolic blood pressure (mmHg) | 71 ± 4 | 70 ± 1 | 0.581 |
| Blood work | | | |
| Triglycerides (mg/dL) | 264 ± 60 | 175 ± 14 | 0.173 |
| Total cholesterol (mg/dL) | 215 ± 17 | 177 ± 47 | 0.046 |
| LDL (mg/dL) | 124 ± 16 | 106 ± 5 | 0.318 |
| HDL (mg/dL) | 35 ± 4 | 39 ± 1 | 0.241 |
| White blood count (1/nL) | 7.8 ± 0.8 | 67.4 ± 0.3 | 0.453 |
| Platelet count (1/nL) | 280 ± 20 | 250 ± 9 | 0.185 |
| Plasma creatinine (mg/dL) | 1.3 ± 0.2 | 1.1 ± 0.1 | 0.325 |
| CRP (mg/L) | 16.7 ± 8.9 | 11.24 ± 1.9 | 0.472 |
| Current medical therapy | | | |
| ASA (%) | 100 | 99 | 1.000 |
| Clopidogrel (%) | 13 | 9 | 0.551 |
| ACE inhibitor (%) | 50 | 68 | 0.432 |
| Calcium antagonist (%) | 13 | 26 | 0.671 |
| β blocker (%) | 63 | 87 | 0.104 |
| Statin (%) | 50 | 64 | 0.466 |
| Oral anti-diabetic drugs (%) | 13 | 17 | 1.000 |
| Insulin (%) | 13 | 23 | 0.676 |
| Current treatment | | | |
| PCI (%) | 13 | 52 | 0.140 |
| Stent (%) | 0 | 21 | 0.096 |
| Prior medical history | | | |
| History of diabetes mellitus (%) | 25 | 57 | 0.457 |
| History of hyperlipidemia (%) | 75 | 77 | 1.000 |
| History of smoking (%) | 75 | 49 | 0.266 |
| Family history of cardiovascular | 63 | 43 | 0.457 |
| disease (%) | | | |
| History of peripheral vascular disease | 38 | 25 | 0.420 |
| (%) | | | |
| Previous myocardial infarction (%) | 25 | 42 | 0.463 |
| Previous CABG (%) | 0 | 16 | 0.593 |
| Previous PCI (%) | 25 | 26 | 1.000 |

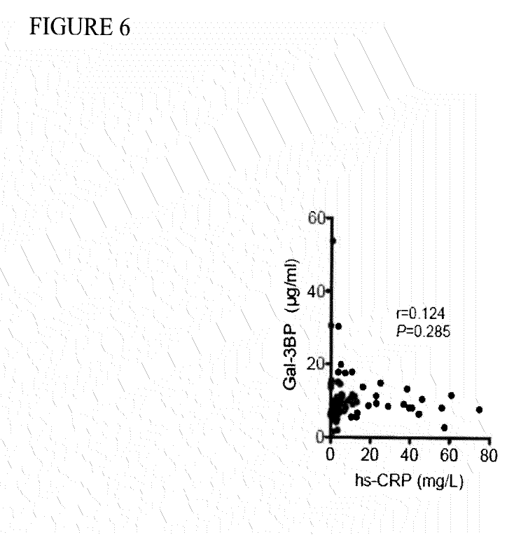

The optimal cut-off determined by ROC analysis was 8.7 μg/ml (area under the curve of 0.73 [95% CI 0.58-0.88; P=0.033]). Sensitivity and specificity were 87.5% and 60.1%, respectively. Kaplan-Meier analysis of this more stringent endpoint demonstrated a highly significant discriminative power with a negative predictive value of 97.1% (P=0.007, hazard ratio 7.024 [95% CI 1.7-29.0]; FIG. 4E). This pattern was similar in patients not treated with intracoronary stent placement. Notably, no correlation between Gal-3BP plasma levels and established risk factors for adverse cardiovascular events including high-sensitivity C-reactive protein was seen (FIG. 6).

For some cardiovascular prognostic markers like high sensitivity C-reactive protein, high levels are associated with elevated risk or poor prognosis (Libby & Ridker, Am. J. Med. 116 Suppl 6A: 9S (2004)). By contrast, elevated Gal-3BP levels predict improved outcome. This may indicate that Gal-3BP expression is upregulated in response to the disease process, resulting in protection from adverse events. The observation that elevated Gal-3BP plasma levels are associated with fewer adverse events is similar to findings reported with adiponectin (Ouchi et al., Circulation 103: 1057 (2001); and Laughlin et al., Am. J. Epidemiol. 165: 164 (2007)).

### Example 6

This example shows Gal-3BP expression within atherosclerotic lesions in human coronary artery atherosclerotic plaques.

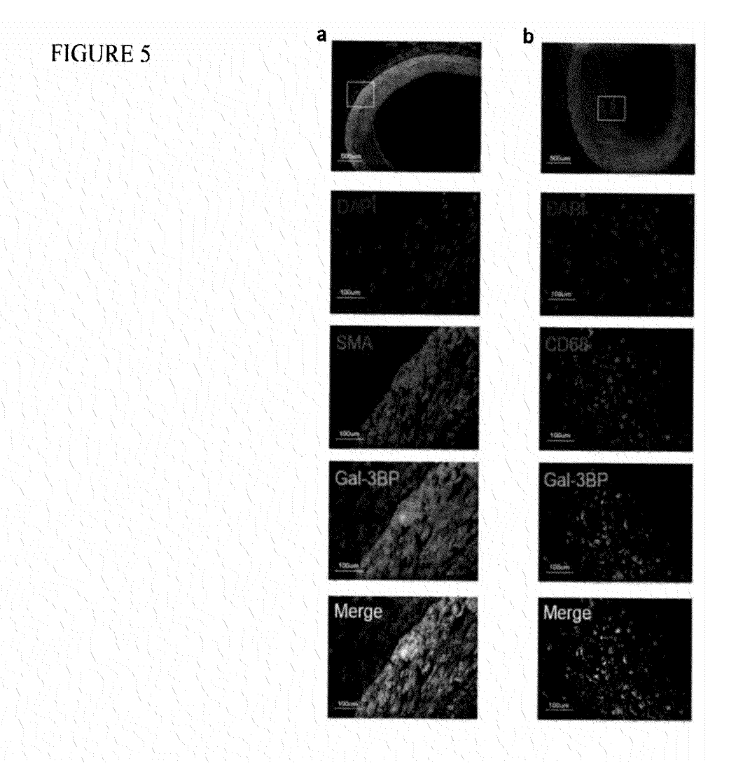

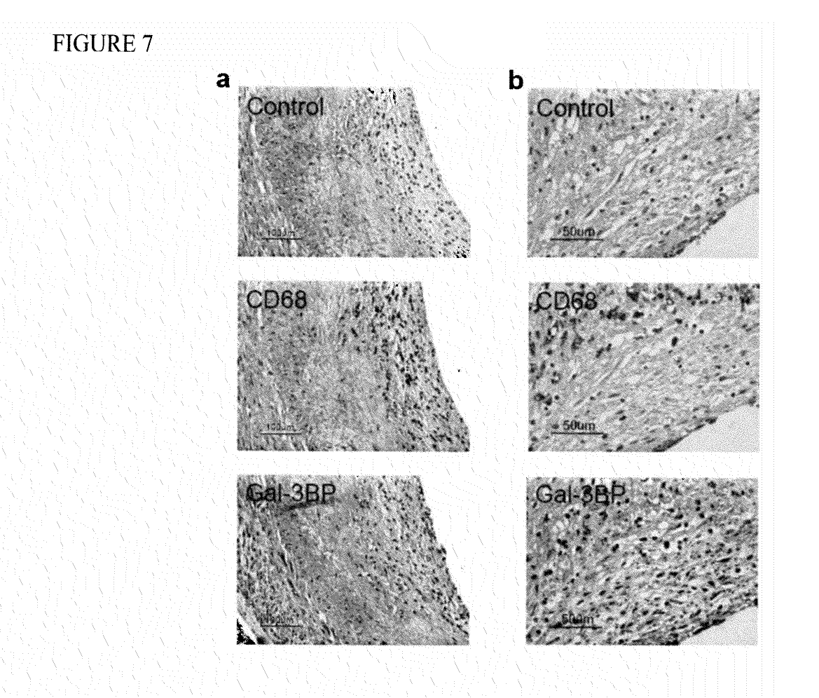

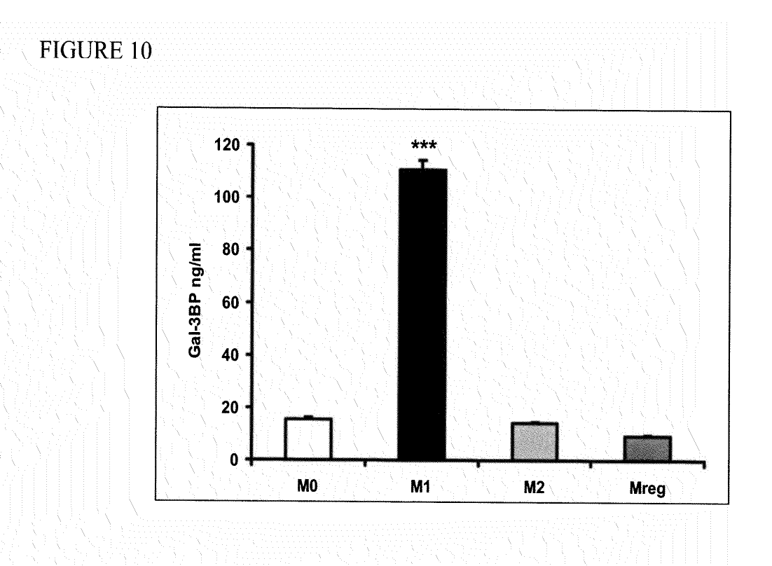

To determine whether Gal-3BP was locally expressed within atherosclerotic lesions, immunoperoxidase staining (FIG. 7) and immunofluorescence (FIG. 5) was used to measure Gal-3BP in human coronary artery atherosclerotic plaques. Positive staining was mainly located in smooth muscle cells (FIG. 5A) and in plaque areas containing CD68⁺ macrophages (FIG. 5B). Not all macrophages were positive for Gal-3BP, suggesting that Gal-3BP may be restricted to a subset of specifically polarized macrophages. In support of this idea, only M1 macrophages differentiated from human blood monocytes were found to secrete significant amounts of Gal-3BP (FIG. 10).

### Example 7

This example shows that Gal-3BP induces a unique macrophage phenotype.

To further characterize the effects of Gal-3BP on macrophages, human blood monocytes were differentiated into M0, M1, M2 and Mreg macrophages, and treated with either LPS or Gal-3BP. Only those cells treated with Gal-3BP produced IL-2, no IL-12 (typical of M1 macrophages), and moderate IL-10 (typical of M2 and Mreg macrophages). These results suggest that Gal-3BP induces a unique macrophage phenotype, characterized in part by production of certain cytokines.

In summary, the data indicates that Gal-3BP provides a protective role in limiting adverse outcomes in atherosclerotic cardiovascular disease. High levels of Gal-3BP reduce macrophage accumulation and uptake of modified LDL in a dose-dependent manner through downregulation of the scavenger receptors CD36 and SR-A. Gal-3BP may thereby affect both plaque composition and stability. Gal-3BP plasma levels may serve as a positive predictor of improved outcome in coronary artery disease.

1. A method of reducing or decreasing risk of an adverse cardiovascular event or cardiovascular disease in a subject, comprising administering a Galectin-3 binding protein (Gal-3BP) polypeptide to a subject in an amount that increases Gal-3BP polypeptide in the subject thereby reducing or decreasing risk of the adverse cardiovascular event or cardiovascular disease in the subject.

2. The method of claim 1, wherein the cardiovascular disease is coronary artery disease, peripheral artery disease, cerebrovascular disease, or renal artery disease.

3. The method of claim 1, wherein the adverse cardiovascular event comprises a stroke, myocardial infarction (heart attack), ischemic heart failure, transient ischemic attack or brain trauma.

4. A method of reducing, inhibiting, or decreasing risk of artherosclerotic plaque formation in a subject, comprising administering a Galectin-3 binding protein (Gal-3BP) polypeptide to a subject in an amount that increases Gal-3BP polypeptide in the subject thereby reducing, inhibiting, or decreasing risk of artherosclerotic plaque formation in the subject.

5. (canceled)

6. The method of any of claims 1 or 4, wherein the Gal-3BP polypeptide increases to an amount in the subject of greater than 2 ug/ml or 5 ug/ml or 10 ug/ml in blood plasma.

7.-10. (canceled)

11. The method of any of claims 1 or 4, wherein the subject has had or is at risk of an adverse cardiovascular event.

12. The method of any of claims 1 or 4, wherein the subject is at risk of or has an existing cardiovascular disease.

13. The method of any of claim 1, 4 or 51 or 4, wherein the Gal-3BP polypeptide comprises a polypeptide fragment of full length Gal-3BP polypeptide.

14. The method of claims 1 or 4, wherein the Gal-3BP polypeptide is mammalian.

15. The method of claims 1 or 4, wherein the Gal-3BP polypeptide is human, Pan troglodytes, Canis lupus familiaris, Bos Taurus, Mus musculus or Rattus norvegicus, SEQ ID NOs:1-6, respectively.

16. The method of claims 1 or 4, wherein the Gal-3BP polypeptide comprises
```
                10 20 30 40 50 60
MTPPRLFWVW LLVAGTQGVN DGDMRLADGG ATNQGRVEIF YRGQWGTVCD NLWDLTDASV
            70 80 90 100 110 120
VCRALGFENA TQALGRAAFG QGSGPIMLDE VQCTGTEASL ADCKSLGWLK SNCRHERDAG
              130 140 150 160 170 180
VVCTNETRST HTLDLSRELS EALGQIFDSQ RGCDLSISVN VQGEDALGFC GHTVILTANL
              190 200 210 220 230 240
EAQALWKEPG SNVTMSVDAE CVPMVRDLLR YFYSRRIDIT LSSVKCFHKL ASAYGARQLQ
              250 260 270 280 290 300
GYCASLFAIL LPQDPSFQMP LDLYAYAVAT GDALLEKLCL QFLAWNFEAL TQAEAWPSVP
              310 320 330 340 350 360
TDLLQLLLPR SDLAVPSELA LLKAVDTWSW GERASHEEVE GLVEKIRFPM MLPEELFELQ
              370 380 390 400 410 420
FNLSLYWSHE ALFQKKTLQA LEFHTVPFQL LARYKGLNLT EDTYKPRIYT SPTWSAFVTD
              430 440 450 460 470 480
SSWSARKSQL VYQSRRGPLV KYSSDYFQAP SDYRYYPYQS FQTPQHPSFL FQDKRVSWSL
              490 500 510 520 530 540
VYLPTIQSCW NYGFSCSSDE LPVLGLTKSG GSDRTIAYEN KALMLCEGLF VADVTDFEGW
              550 560 570 580
KAAIPSALDT NSSKSTSSFP CPAGHFNGFR TVIRPFYLTN SSGVD
```

17. The method of claims 15 or 16, wherein the Gal-3BP polypeptide fragment is residues 24-124 (SRCR domain); residues 153-221 (BTB domain); or residues 260-360 (BACK domain).

18. The method of any of claims 1 or 4, wherein the Gal-3BP polypeptide comprises a fusion polypeptide, or a chimeric polypeptide.

19. The method of claim 18, wherein the chimeric polypeptide comprises a Gal-3BP/Immunoglobulin fusion polypeptide.

20.-29. (canceled)

30. The method of any of claims 1 or 4, wherein the subject has or is a candidate for a blood cholesterol lowering therapy.

31.-40. (canceled)

41. A method of reducing or inhibiting foam cell formation in a subject, comprising administering a Galectin-3 binding protein (Gal-3BP) polypeptide to a subject in an amount that increases Gal-3BP polypeptide in the subject thereby inhibiting or reducing foam cell formation in the subject.

42. (canceled)

43. An isolated or purified subsequence of full length Galectin-3 binding protein (Gal-3BP) polypeptide, wherein the subsequence reduces or inhibits macrophage cell expression of scavenger receptor A or CD36, reduces or inhibits foam cell formation or reduces or inhibits modified LDL uptake by macrophages.

44. A pharmaceutical composition comprising Galectin-3 binding protein (Gal-3BP) polypeptide or a subsequence of full length Galectin-3 binding protein (Gal-3BP) polypeptide, wherein the Galectin-3 binding protein (Gal-3BP) polypeptide or subsequence reduces or inhibits macrophage cell expression of scavenger receptor A or CD36, reduces or inhibits foam cell formation or reduces or inhibits modified LDL uptake by macrophages.

45. The subsequence of Galectin-3 binding protein (Gal-3BP) polypeptide of claim 43 or 44, wherein the subsequence consists of BTBD17B residues 24-124 (SRCR domain); residues 153-221 (BTB domain); residues 260-360 (BACK domain), or a subsequence of BTBD17B residues 24-124 (SRCR domain); residues 153-221 (BTB domain); residues 260-360 (BACK domain).

46. The subsequence of claims 43 or 44, wherein the subsequence of Gal-3BP residues 24-124 (SRCR domain); residues 153-221 (BTB domain); or residues 260-360 (BACK domain) is about 5-10, 10-20, 20-50, 50-75 or 50-100 amino acids in length.

47. The subsequence of Galectin-3 binding protein (Gal-3BP) polypeptide of claims 43 or 44, wherein the subsequence comprises a fusion construct.

48. The subsequence of claim 47, wherein the fusion construct comprises a chimeric polypeptide.

49. The subsequence of Galectin-3 binding protein (Gal-3BP) polypeptide of claims 49 or 50, wherein the subsequence is about 5-10, 10-20, 20-50, 50-100, 100-150, 150-200, 200-250, 250-300, 300-350, 350-400, 400-500, 500-600 or more amino acids in length, and less than full length Gal-3BP polypeptide sequence.

50. A method of diagnosing a subject having or at increased risk of having an adverse cardiovascular event or cardiovascular disease, artherosclerotic plaque formation, foam cells or foam cell formation, comprising contacting a biological material or sample from a subject with an agent that binds to Gal-3BP polypeptide sequence and assaying for the amount of Gal-3BP polypeptide, wherein an amount less than about 10 ug/ml diagnoses the subject as having or at increased risk of developing an adverse cardiovascular event, cardiovascular disease, artherosclerotic plaque formation, foam cells or foam cell formation.

51. The method of claim 50, wherein the agent comprises an antibody that binds to Gal-3BP polypeptide or a nucleic acid that hybridizes to a nucleic acid encoding Gal-3BP polypeptide sequence.

52. The method of claim 50, wherein the cardiovascular disease comprises coronary artery disease, peripheral artery disease, cerebrovascular disease, or renal artery disease.

53. The method of claim 50, wherein the adverse cardiovascular event comprises stroke, myocardial infarction (heart attack), ischemic heart failure, transient ischemic attack or brain trauma.

54. The method of claim 50, wherein the subject is human.

55. The method of claim 50, wherein the sample is blood or plasma.
